(19) United States
(12) Patent Application Publication
(10) Pub. No.: US 2005/0260588 A1
(43) Pub. Date: Nov. 24, 2005

(54) In vivo ssdna expression vectors for altering gene expression

(76) Inventors: Charles A. Conrad, Houston, TX (US); Yin Chen, Pearland, TX (US)

Correspondence Address: Mark R Wisner;Wisner & Associates, 1177 West Loop South, Suite 400, Hoston, TX, 77027-9012, US

(21) Appl. No.: 10/513,191

(22) PCT Filed: May 1, 2003
(86) PCT No.: PCT/US03/13593

§ 371 Date: Jul. 11, 2005

(30) Foreign Application Priority Data
May 1, 2002 (US) 10136218

(51) Int. Cl. C12Q 1/68; C12P 21/06; C12N 15/74

(52) U.S. Cl. 435/6; 435/69.1; 435/320.1; 435/325

(57) ABSTRACT

An expression vector for altering expression of a target nucleic acid sequence in a host cell by production of single-stranded cDNA (ssDNA) in the host cell in vivo. The expression vector is comprised of a cassette comprising a sequence of interest, an inverted tandem repeat, and a primer binding site 3′ to the inverted tandem repeat, and a reverse transcriptase/RNAse H coding gene, and may be transfected into the host cell. Transcription of the cassette by the host cell produces an RNA template which is reverse transcribed with the product of the RT coding gene to produce ssDNA of a specified sequence. The ssDNA is modified to remove flanking vector sequences by taking advantage of the “stem-loop” structure of the ssDNA, which forms as a result of the inverted tandem repeat that allows the ssDNA to fold back on itself, forming a double stranded DNA stem. The double-stranded stem may contain one or more restriction endonuclease recognition sites and the loop, which remains as ssDNA, can be any desired nucleotide sequence. This design allows the double-stranded stem of the stem-loop intermediate to be cleaved by the desired corresponding restriction endonuclease(s) and the loop portion is then released as a linearized, single-stranded piece of DNA. The resulting ssDNA binds to an endogenous target nucleic acid sequence to alter the expression of that sequence for such therapeutic purposes as gene activation or inactivation using duplex or triplex binding of nucleic acids, site-directed mutagenesis, interruption of cellular function by binding to specific cellular proteins, or interfering with RNA splicing functions.

The present invention relates to the production of ssDNA in vivo. More particularly, the present invention relates to a system for delivering the information required for production of ssDNA in vivo for such purposes of altering gene function and for expressing that information in vivo. The information that is delivered and subsequently expressed in vivo includes (a) a sequence of interest (SOI) that codes for the ssDNA sequence and (b) the signaling instructions and enzymatic function(s) for producing that ssDNA sequence in vivo. Delivery is accomplished by incorporating the SOI, and the signaling instructions and enzymatic functions, into a viral vector such as an adenoviral vector or by constructing a plasmid containing the SOI, and the signaling instructions and enzymatic functions, and packaging that plasmid into a liposome or other vehicle for delivery to a prokaryotic or eukaryotic host cell. Regardless of whether the information is delivered and/or expressed-by a viral vector, by a plasmid and delivery vehicle, or by other mechanism, the phrase “expression vector” is utilized for the purpose of referring to the system for delivering and expressing the information that causes a change in gene function in the host cell.

More specifically, the expression vector comprises a cassette into which a nucleic acid sequence is incorporated for use as a template for production of that sequence in a prokaryotic or eukaryotic host cell, and subsequent expression within prokaryotic or eukaryotic host cells, as a single stranded DNA (ssDNA) sequence without (or with minimal) flanking sequences that binds to or otherwise interacts with a target gene to alter expression of the target gene. The expression vector of the present invention removes most or all contiguous plasmid (or other vector) sequences from the ssDNA either by stem-loop formation with subsequent termination of a reverse transcription reaction by the stem or by cleavage of the stem-loop intermediate. The ssDNA is designed to be complimentary to and/or to otherwise bind to any endogenous nucleic acid sequence target, thereby targeting any desired gene.

There has been increasing interest in oligonucleotides (ODNs) as tools for understanding gene function by knock-out or investigating knock-down target genes, for validating new genomic drug targets, and ultimately as potential therapeutic agents. As used in this specification, the term “ODN's” is intended to refer to: 1) DNA-based oligonucleotides such as triplex-forming oligonucleotides (TFO), antisense ODN's, DNA enzymes and aptamers and 2) RNA-based oligonucleotides such as ribozymes. All of these molecules alter gene expression by interacting with DNA or mRNA in sequence-specific manner. A number of ODN-based drugs have already entered advanced clinical trials. Uhlman, E., Oligonucleotide technologies: Synthesis, production, regulations and application, 1 Exp. Opin. Biol. Ther., 319-328 (2001). Vitravene™, the first ODN-based drug, is now marketed for the treatment of cytomegalovirus retinitis infections in AIDS patients.

Antisense gene therapy has been used in a variety of applications to regulate gene function. Jain, K. K., Handbook of Gene Therapy, New York: Hofgrefe & Huber Publishing (1998). To date, however, such therapy has been characterized by a number of disadvantages and limitations that decrease the utility of this type of therapy, including the short half-life of the antisense molecule in vivo, non-specific effects, uncertainties as to the mode of action of the antisense sequence, and potential toxic effects. For instance, antisense oligonucleotides (ODNs) and their analogs must be administered intravenously, which involves problems in cell uptake and distribution (Cossum, P. A., et al., Disposition of the ¹⁴C-labeled phosphorothioate oligonucleotide ISIS 2105 after intravenous administration to rats, 267 J. Pharmacol. Exp. Ther. 1181-1190 (1993), Sands, H., et al., Biodistribution and metabolism of internally ³H-labeled oligonucleotides. II. 3′, 5′-blocked oligonucleotides, 47 Mol. Pharmacol. 636-646 (1995)) as well as toxicity problems due to high blood concentrations (Henry, S. P., et al., Evaluation of the toxicity of ISIS 2302, a phosphorothioate oligonucleotide, in a 4-week study in CD-1 mice, 7 Antisense Nucleic Acid Drug Dev. 473-481 (1997), Henry, S. P., et al., Comparison of the toxicity profiles of ISIS 1082 and ISIS 2105, phosphorothioate oligonucleotides, following subacute intradermal administration in Sprague-Dawley rates, 116 Toxicology 77-88 (1997)).

The antisense ODN analogs used most in antisense therapies are phosphorothioates or methylphosphonates. However, phosphorothioate ODNs tend to bind serum and intracellular proteins nonspecifically (Crooke, S.T., et al., Pharmocokinetic properties of several novel oligonucleotide analogs in mice, 227 J. Pharmacol. Exp. Ther. 923-937 (1996), Gao, W.Y., et al., Phosphorothioate oligonucleotides are inhibitors of human DNA polymerases and RNase H: implications for antisense technology, 41 Mol. Pharmacol. 223-229 (1992)), and at higher concentrations, inhibit RNase H activity (Crooke, S.T., el al., Kinetic characteristics of Escherichia coli RNase H: Cleavage of various antisense oligonucleotide-RNA duplexes, 312 Biochem. J. 599-608 (1995)). Phosphorothioate ODNs have a lower Tm (an average of 0.5° C. per base pair) for RNA than does natural DNA (Crooke, S. T. and B. LeBleu, Antisense research and application, Boca Raton: CRC Press (1993)). This lower Tm requires that phosphorothioate ODNs typically be longer than phosphodiester DNA oligonucleotides for effective binding. However, an increase in the length of the ODN can cause a loss of hybridization specificity (Toulme, J. J., et al., Antisense technology: A practical approach, in C. Lichtenstein and W. Nellen (Eds.), New York: IRL Press, pp. 39-74 (1997)). In addition, methylphosphonate ODNs do not activate RNase H enzyme activity (Maher, L. J, et al., Inhibition of DNA binding proteins by oligonucleotide-directed triple helix formation, 245 Science 725-730 (1989), Miller, P. S., Oligodeoxynucleotides: Antisense inhibitors of gene expression, in J. S. Cohen (Ed.), Boca Raton: CRC Press, p. 79 (1989)) and are eliminated rapidly (Chen, T. L., et al., Disposition and metabolism of oligodeoxynucleoside methylphosphonate following a single i.v. injection in mice., 18 Drug Metab. Dispos. 815-818 (1990)). For a summary of the many strategies that have been attempted for delivery of ODNs, and their varying degrees of success, see Akhar, S., el al., The delivery of antisense therapuetics, 44 Adv. Drug Delivery Rev. 3-21 (2000).

Another approach to gene therapy is to administer molecules that have catalytic activity against a target gene and/or the transcriptional product of the target gene. For instance, ribozymes are capable of catalyzing the cleavage of specific mRNA sequences, and are thought to be potentially more efficient in targeting the target gene than antisense ODNs because of their catalytic capability. Wool, T. M., To cleave or not to cleave: Ribozymes and antisense, 5 Antisense Res. Dev. 227-232 (1995). Ribozymes have been used as inhibitors of gene expression and viral replication. Jain, supra (1998). Unlike antisense ODNs, ribozymes can be delivered either endogenously, such as by using viral vectors, or exogenously. However, ribozymes have limited stability due to degradation by RNases in vivo. Jain, supra (1998).

Using in vitro selections, several small single-stranded DNAs have been demonstrated to catalyze RNA cleavage (Breaker, R. R., Catalytic DNA: In training and seeking employment, 17 Nature Biotechnology 422-423 (1999)), thereby offering the promise of targeted, gene specific activity. The patent and scientific literature describes a number of deoxynucleic acid sequences with known catalytic activity (see, Breaker, R. R. and G. F. Joyce, I Chem. Biol. 223-229 (1994); Cuenoud, B. and J. W. Szostak, 375 Nature 611-613 (1995); Santoro, S. W. and G. F. Joyce, 94 Proc. Natl. Acad. Sci. USA 4262-4266 (1997); Faulhammer and M. Famulok, 269 J: Molec. Bio. 188-203 (1997); Carmi, N, etal., 95 Proc. Natl. Acad. Sci. USA (1998); Li, Y. and R. R. Breaker, 96 Proc. Natl. Acad. Sci. USA 2746-2751 (1999) and U.S. Pat. Nos. 5,807,718 and 5,910,408), including the so-called “10-23 DNA enzyme” and other ssDNA sequences that act, for instance, as copper-dependent DNA ligases and calcium-dependent DNA kinases. The catalytic efficiency of such sequences has been demonstrated for cleaving mRNA targets at 10⁹ m⁻¹/min⁻¹ in the presence of divalent magnesium, thereby offering the opportunity for targeted destruction of substrate molecules (see, for instance, R. R. Breaker, supra (1999)).

Although the art appears to recognize the potential for use of this enzymatic activity for therapeutic purposes, so far as is known, no system is available for producing target-specific enzymatic nucleic acid sequences to produce a therapeutic effect in vivo. Consequently, one object of the present invention is to provide a DNA expression vector that delivers the information to a target cell that directs the synthesis of ssDNA containing a sequence that specifically cleaves specified MRNA target(s) in vivo to alter the expression of the gene producing that target mRNA(s).

Because secondary structure folding may be critical to the catalytic function of the enzymatic sequence of the ssDNA, it is another object of the present invention to produce ssDNA including a DNA enzyme sequence of any desired nucleotide sequence within eukaryotic cells without intervening or flanking nucleotide bases to preserve the enzymatic function of the ssDNA against a target nucleic acid for altering the expression of a gene including the target nucleic acid.

Another object of the present invention is to provide an expression vector for producing ssDNA of any nucleotide sequence in vivo that functions as (but is not limited to) an inhibitory nucleic acid for, for instance, binding to one or more mRNAs in anti-sense fashion, to down regulate a gene product or a viral gene product of interest or binding to and inhibiting a specific cellular function, for instance, by binding to proteins that recognize a nucleic acid sequence.

Another object of the present invention is to provide an expression vector for producing ssDNA of any nucleotide sequence in vivo that functions as (but is not limited to) an excitatory nucleic acid for, for instance, binding to one or more target endogenous DNA sequences to increase production of or to “switch on” a target gene.

Another object of the present invention is to provide an expression vector for producing ssDNA designed to favor binding to duplex (native DNA) to form triplex structures that interfere with normal gene transcription and regulation of a target gene.

Another object of the present invention is to produce ssDNA within eukaryotic cells for the purpose of disrupting and/or altering one or more cell functions.

Yet another object of the present invention is to provide an expression vector for producing ssDNA into which secondary structures are designed so that the ODN's produced by the vector bind to and/or otherwise inhibit or activate various cellular functions that rely on the catalytic action of a protein or on nucleic acid protein interaction such as transcription, translation, and DNA replication.

Another object of the present invention is to provide an expression vector for producing ssDNA in vivo for site-directed mutagenesis or gene knockout for therapeutic applications.

Another object of the present invention is to provide an expression vector for producing ssDNA of precisely defined nucleotide composition that favors site-specific insertion into a genome for therapeutic purposes.

Yet another object of the present invention is to provide an expression vector for producing ssDNA that is complimentary to any endogenous nucleic acid target for use in altering expression of a gene including the nucleic acid sequence target.

Another object of the present invention is to provide an expression vector for in vivo production of ssDNA including an inhibitory or excitatory sequence against DNA and/or mRNA targets for introduction into prokaryotic or eukaryotic cells that overcomes the disadvantages of direct administration of ssDNA by lipofection, direct cellular uptake, and/or microinjection.

Another object of the present invention is to provide an expression vector for in vivo production of ssDNA including a sequence exhibiting catalytic activity against mRNA targets for introduction into prokaryotic and eukaryotic cells using liposomal or viral delivery vehicles, electroporation, or related means for targeting specific cells.

Another object of the present invention is to provide all enzymatic functions needed to produce an inhibitory or excitatory ssDNA sequence in vivo with activity against a target mRNA or DNA sequence of choice in a single plasmid.

Another object of the present invention is to provide pharmacologically acceptable compositions for delivering inhibitory or excitatory nucleic acid sequences in a manner that produces a therapeutic effect.

This listing of the objects of the present invention is not intended to be a list of all the objects of this invention. There are many cellular functions that are 25 mediated by the cellular genome which, in the interest of brevity and practicality, are not mentioned here and which are amenable to regulation by in vivo production of ssDNA. For instance, exonucleases digest ssDNA much more aggressively than double-stranded DNA (dsDNA). Consequently, another object of the present invention is to provide an expression vector for producing nucleic acid sequences in vivo that are not as susceptible to degradation by native exonucleases in the cell as double-stranded DNA. It can be seen from this illustration that this list of objects of the present invention is provided for exemplification and is not intended to limit the scope of the invention.

These objects are provided by an expression vector for use in producing ssDNA in a host cell that binds to or otherwise interacts with an endogenous nucleic acid target sequence in that target cell comprising a cassette comprised of a sequence of interest flanked by an inverted tandem repeat, a 3′ primer binding site (PBS), and a gene encoding a reverse transcriptase for transcribing the mRNA transcript of the cassette from the PBS to release a single-stranded cDNA transcript in the cell. The sequence of interest is comprised of a nucleic acid sequence that produces a sequence of nucleic acids that binds to or otherwise interacts with an endogenous target nucleic acid sequence when reverse transcribed to alter expression of the target sequence.

Several embodiments of the invention are illustrated in the figures, in which

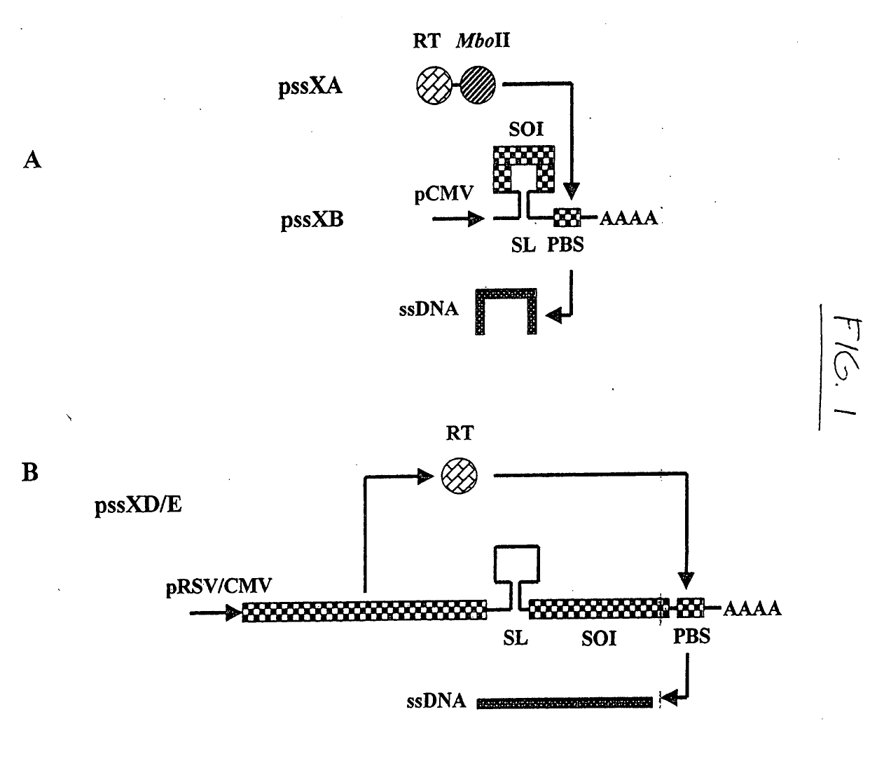

FIG. 1 is a schematic illustration of a production of ssDNA in a host cell in accordance with the present invention.

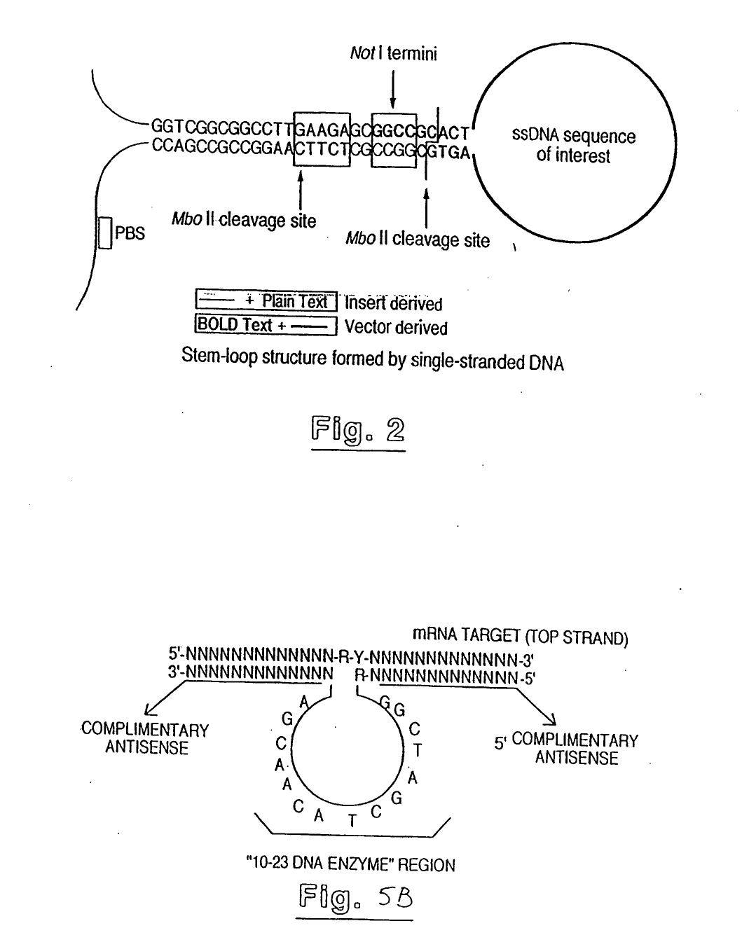

FIG. 2 is a schematic illustration of the stem-loop intermediate formed by the method illustrated in FIG. 1.

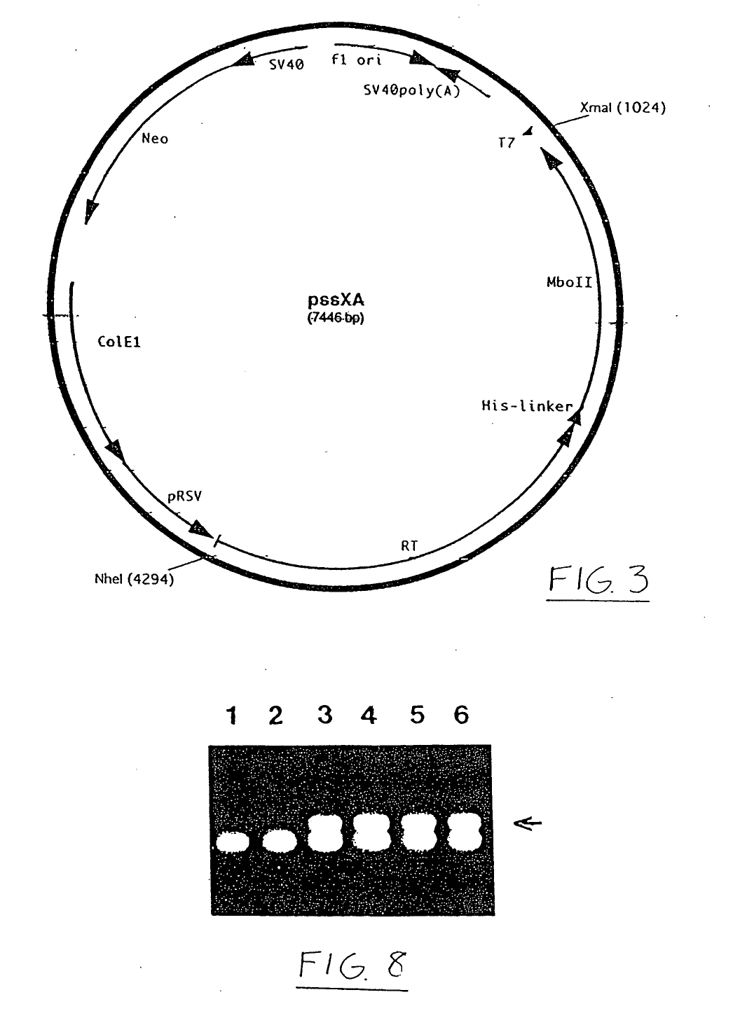

FIG. 3 is a schematic illustration of the pssXA plasmid comprising a first component of a first embodiment of the expression vector of the present invention. To make pssXA, reverse transcriptase (RT) and MboII genes were subcloned into the mammalian expression vector pBK-RSV (Stratagene) and expressed as a single polypeptide. The RT and MboII domains are separated by a histidine-rich linker.

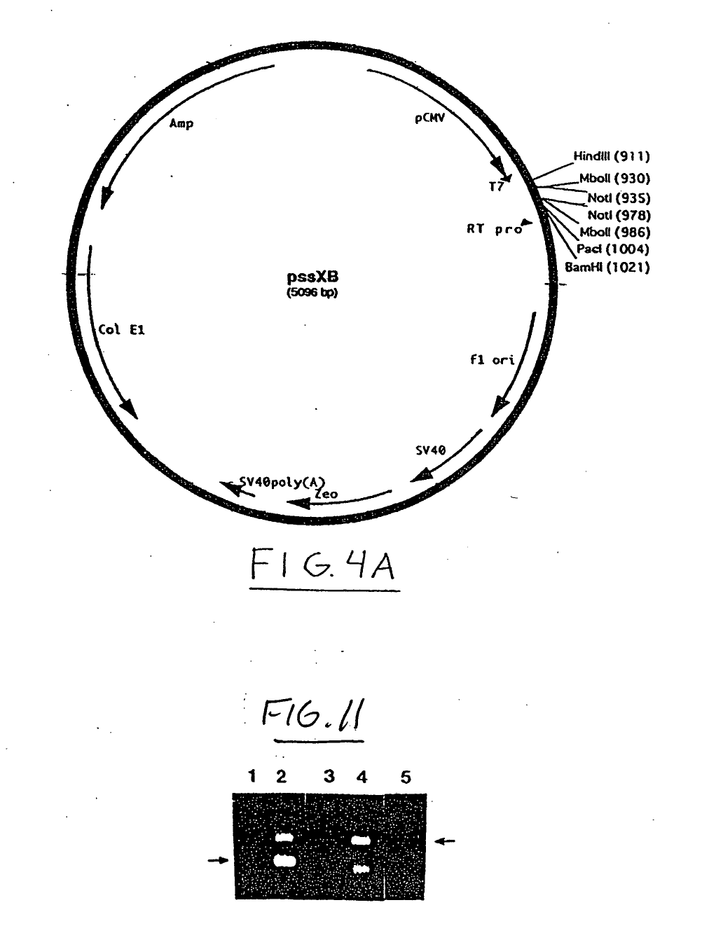

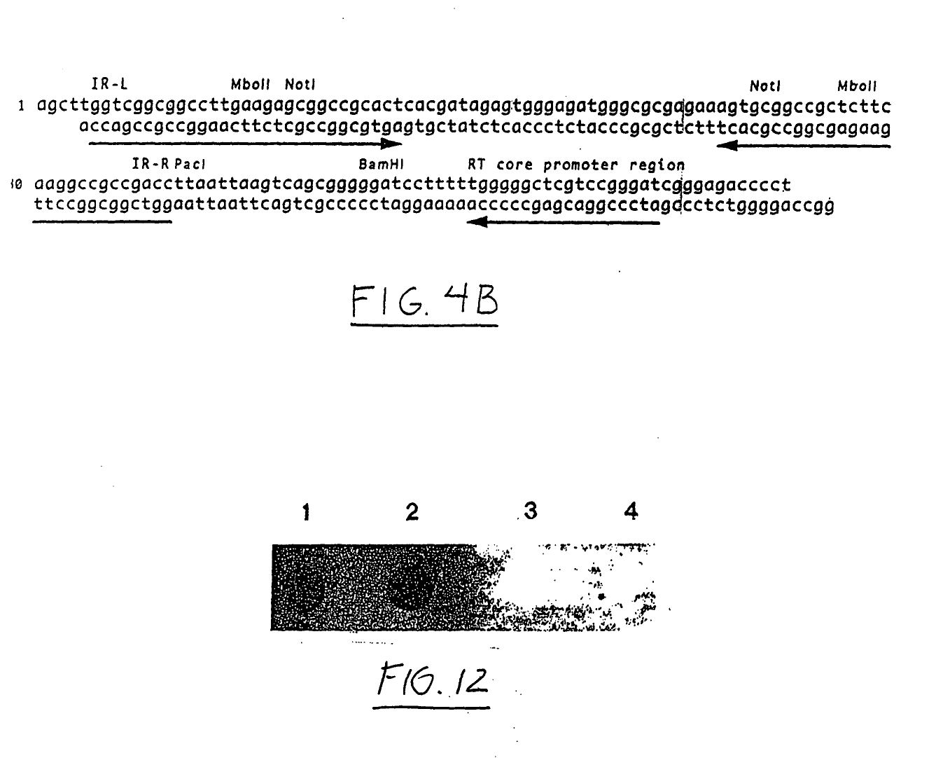

FIGS. 4A and 4B are schematic illustrations of the pssXB plasmid comprising a second component of the first embodiment of the expression vector of the present invention. As shown in Fig: 4A, the pssXB plasmid includes a sequence of interest and (1) the MoMuLV reverse transcriptase promoter region, (2) two NotI, one PacI, and one BamHI sites for subcloning a DNA sequence of interest, and (3) the tandem inverted repeats, IR-L and IR-R. The sequence of the insert region of the pssXB plasmid is shown in FIG. 4B.

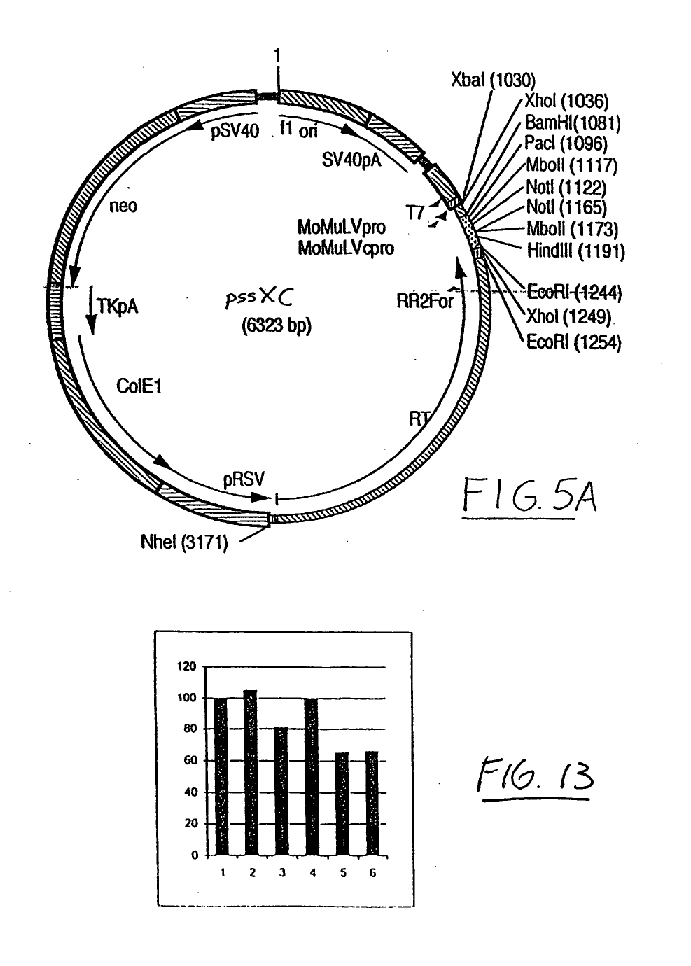

FIG. 5A is a schematic illustration of the pssXC plasmid comprising a second embodiment of the expression vector of the present invention that includes the 10-23 DNA enzyme sequence illustrated schematically in FIG. 5B.

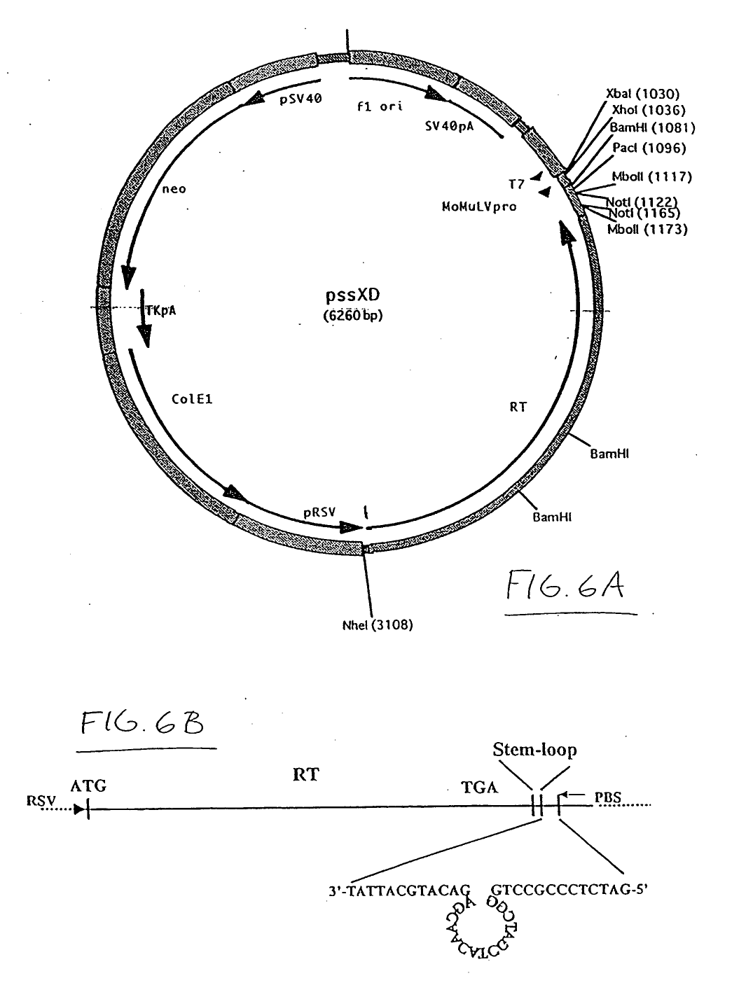

FIG. 6A represents a schematic illustration of the pssXD plasmid comprising a third embodiment of the expression vector of the present invention, with an enlarged portion of the pssXD plasmid being shown in FIG. 6B.

FIG. 7 represents a schematic illustration of the pssXE plasmid comprising a fourth embodiment of the expression vector of the present invention.

FIG. 8 shows the result of a PCR assay for RT activity in a pssXA transfected cell lysate. Lanes 1 and 2: A549 cells transiently transfected with the pBK-RSV vector; lanes 3 and 4: A549 cells transiently infected with pssXA; lanes 5 and 6: A549 cells stably transfected with pssXA (E10). Before PCR amplification, reverse transcription reaction was carried out for 10 (lane 1, 3, and 5) or 30 minutes (lane 2, 4, and 6), respectively, at 37° C.

FIG. 9 represents a schematic illustration of the pssXF plasmid comprising a fifth embodiment of the expression vector of the present invention.

FIG. 10 represents a schematic illustration of the pssXV plasmid comprising a sixth embodiment of the expression vector of the present invention.

FIG. 11 shows the result of an assay for detecting ssDNA by PCR analysis. Total RNA isolated from either EIO cells, transiently transfected with pssXB vector,. pssXB-I or pssXB-II. Before PCR amplification, total RNA was pre-treated with either S1 nuclease (lanes 1 and 3) or RNase (lanes 2, 4, and 5) for 30 minutes at 37° C. lanes 1 and 2: pssXB-I; lanes 3 and 4: pssXB-II; lane 5: pssXB vector.

FIG. 12 shows the results of a dot blot analysis for detection of ssDNA. 1: E10 cells transfected with pssXB-I; 2. E10 cells transfected with pssXB-II; 3: E10 cells; 4: A549 cells.

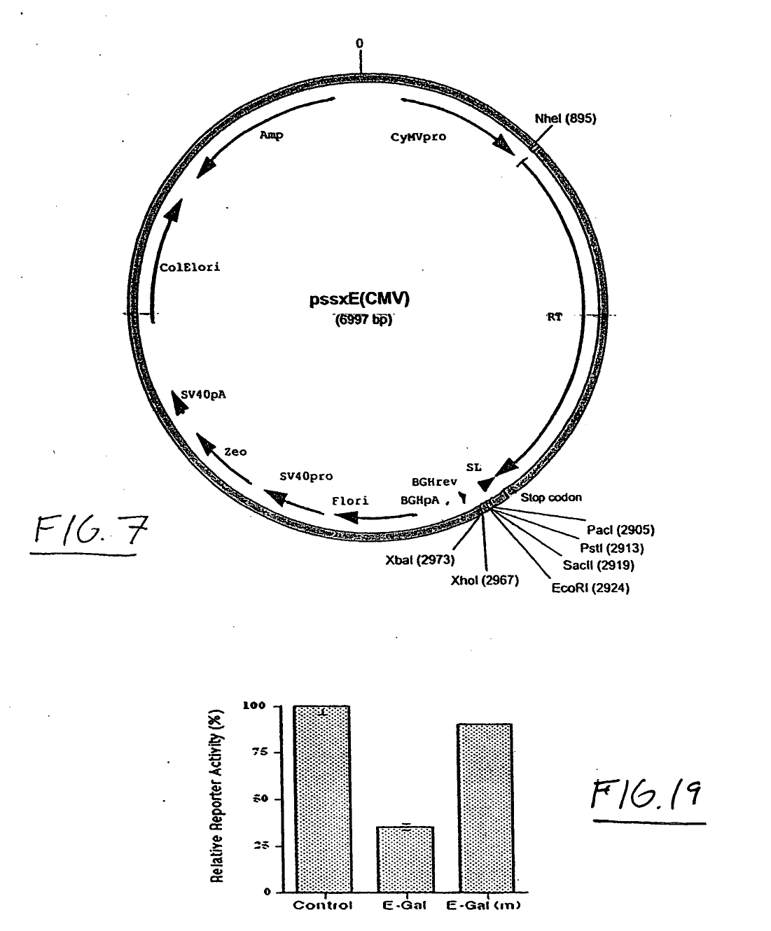

FIG. 13 shows a bar graph quantitating a Northern blot of a ssDNA-producing vector constructed in accordance with the present invention producing an antisense sequence against c-raf kinase. Lanes 1-3: cells harvested 24 hrs after transfection; lanes 4-6: cells harvested 48 hrs after transfection. Lane 1: E10 cells transfected with pssXB vector; lanes 2 and 5: E10 cells transfected with pssXB-I; lanes 3 and 6: E10 cells transfected with pssXB-II.

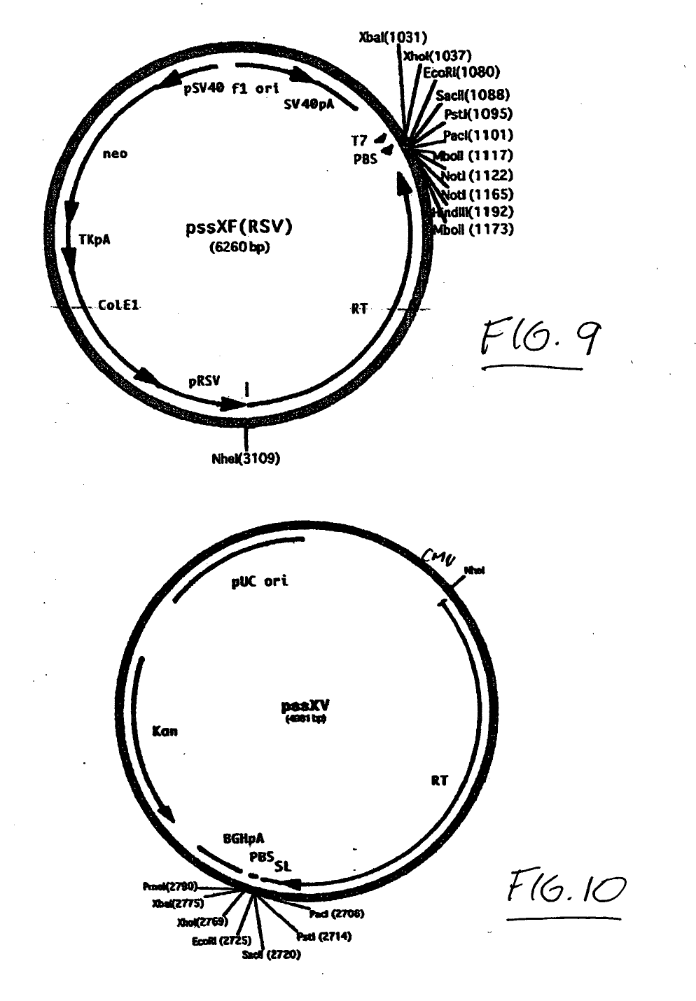

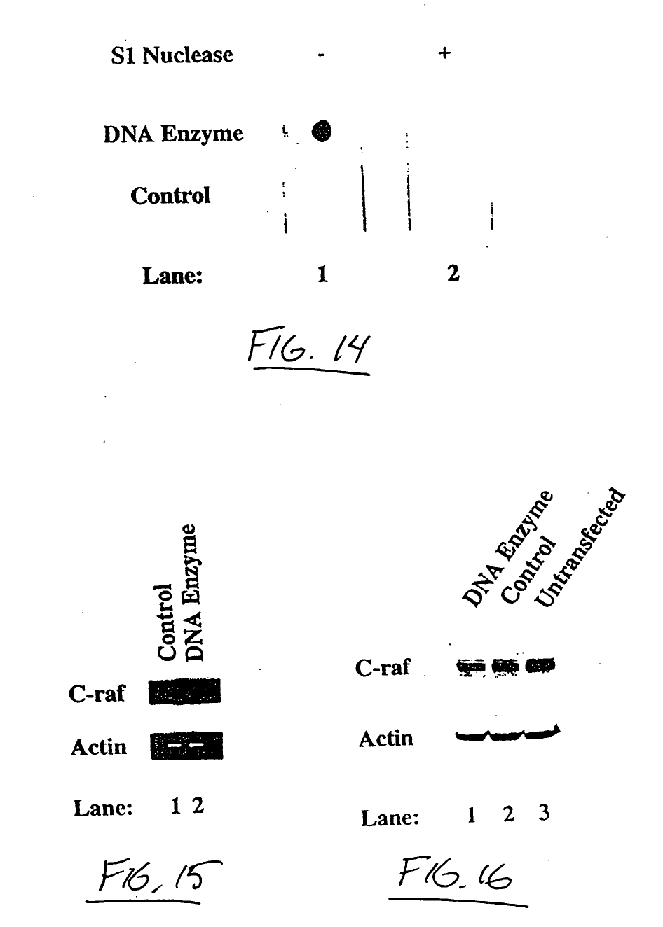

FIG. 14 shows the results of a dot blot analysis for detection of ssDNA in A549 cells transfected with control pssXD-I or pssXD-II containing the c-raf DNA enzyme sequence. No detectable signal was produced in the presence of S1 nuclease due to the specific degradation of ssDNA enzyme by S1 nuclease.

FIG. 15 shows the results of quantitative RT-PCR to determine whether ssDNA expressed in A549 cells transfected with pssXD-II altered c-raf mRNA levels. Lane 1: control pssXD-I; Lane 2: pssXD-II.

FIG. 16 shows the results of a Western blot for suppression of c-raf protein expression in A549 cells transfected with pssXD-I or pssXD-II. Lane 1: pssXD-II; Lane 2: control pssXD-I; Lane 3: untransfected cells.

FIG. 17 shows the results of a Western blot for genomic DNA cleavage for induction of cell apoptosis by suppression of c-raf gene expression. Lane 1: pssXD-II; Lane 2: control pssXD-I; Lane 3: untransfected cells.

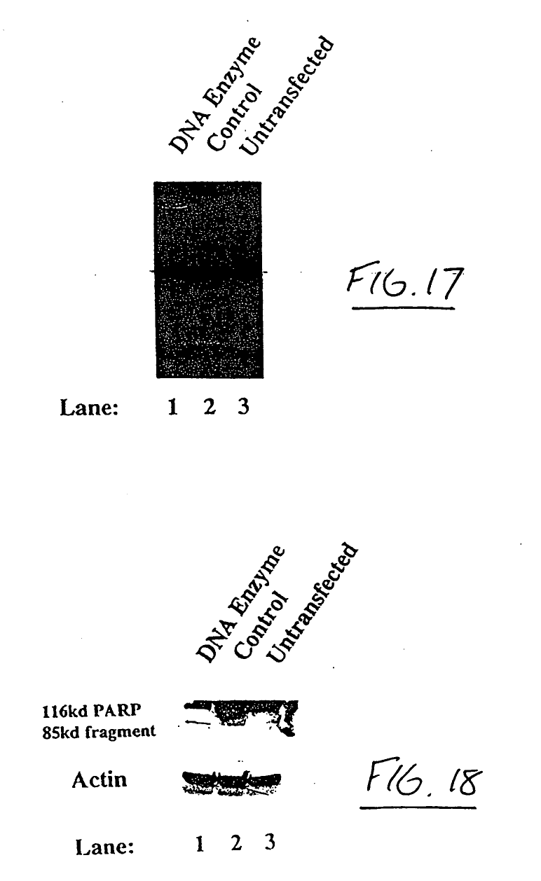

FIG. 18 shows the results of a Western blot for PARP cleavage for induction of cell apoptosis by suppression of c-raf gene expression. Lane 1: pssXD-II; Lane 2: control pssXD-I; Lane 3: untransfected cells.

FIG. 19 shows the inhibition of B-Gal expression by in vivo produced DNA enzyme targeting mRNA.

In more detail, an expression vector is described for use in producing single-stranded deoxyribonucleic acid (ssDNA) oligonucleotides (ODN's) of virtually any predefined or desired nucleotide base composition iii vivo in yeast, prokaryotic cells, and/or eukaryotic cells, with or without flanking nucleotide sequences, for use in altering the expression of a target gene. In one embodiment, the expression vector of the present invention (as used herein, the term “vector” refers to one or more plasmids or modified viral or non-viral recombinant biological constructs used to deliver and manipulate synthesized and/or naturally occurring nucleic acid sequences) is designed to produce a sequence of interest as a ssDNA molecule within mammalian cells. The vector contains all the necessary enzymatic functions and signaling instructions for producing ssDNA in the host cell. As shown in FIG. 1 (illustrating the use of a plasmid as an expression vector constructed in accordance with the teachings of the present invention), the host cell produces an RNA transcript, driven by an eukaryotic promoter, that is used as a template to direct synthesis of the desired ssDNA sequence

In a first embodiment shown in FIG. 1A, the expression vector of the present invention comprises two plasmids that are co-transfected into yeast or any prokaryotic or eukaryotic host cell to produce a ssDNA sequence in the cell for altering gene expression. In a second embodiment, the expression vector comprises a single plasmid (Fig. 1B) including the sequence of interest that is transfected into a host cell for production of the ssDNA sequence of interest for altering gene expression. The in vivo ssDNA may be any ODN, including ODN's that function as inhibitory or excitatory nucleic acids. Inhibitory nucleic acids may be ssDNA synthesized from the mRNA template, or the MRNA template itself, which can specifically bind to a complementary nucleic acid sequence in the host cell. By binding to the appropriate target nuclei. acid sequence, an RNA--RNA, a DNA-DNA, or RNA--DNA duplex or triplex is formed. More commonly, these nucleic acid sequences are termed “antisense” sequences because they are usually complementary to the sense, or coding strand of the gene, but the “sense” sequence is also utilized in the cell for therapeutic purposes. The phrases “inhibitory nucleic acids” and “excitatory nucleic acids” as used herein, therefore, include both “sense” and “antisense” nucleic acids, but as set out below, these phrases are not intended to be limited to sense or antisense-nucleic acids.

By binding to a target nucleic acid, an inhibitory/excitatory nucleic acid alters the function of the target nucleic acid. This alteration (usually an inhibitory effect) results from, for example, blocking DNA transcription, processing or poly(A) addition to mRNA, DNA replication, translation, or promoting inhibitory mechanisms of the cells (such as promoting RNA degradation). Inhibitory nucleic acid methods therefore encompass a number of different approaches, functioning in several different ways, to alter gene expression. Because of the many ways in which they function to alter gene function, broad reference is made herein to binding, or otherwise interacting with, the target gene. The different types of inhibitory nucleic acid technologies are described in Helene, C. and J. Toulme (1049 Biochem. Biophys. Acta. 99-125 (1990)), hereinafter referred to as “Helene and Toulme,” which is incorporated herein in its entirety by this specific reference thereto.

In brief, inhibitory nucleic acid therapy approaches can be classified into (1) hose that target DNA sequences, (2) those that target RNA sequences (including pre-mRNA and mRNA), (3) those that target proteins (sense strand approaches), and (4) those that cause cleavage or chemical modification of the target nucleic acids such as the ssDNA enzymes, including the so-called “10-23 enzyme” as described herein. The first approach contemplates several categories. Nucleic acids are designed to bind to the major groove of the duplex DNA to form a triple helical or “triplex” structure. Alternatively, inhibitory/excitatory nucleic acids are designed to bind to regions of ssDNA resulting from the opening of duplex DNA during replication or transcription. More commonly, inhibitory/excitatory nucleic acids are designed to bind to mRNA or mnRNA precursors. Inhibitory nucleic acids are also designed to prevent maturation of pre-mRNA or to interfere with RNA processing, splicing or translation. Using this second approach, the inhibitory nucleic acid is used to selectively alter certain cellular functions by inhibition/excitation of translation of mRNA encoding critical proteins. An example of an inhibitory nucleic acid is the sequence that is complementary to regions of c-myc MRNA, which inhibits c-myc protein expression in a human promyelocytic leukemia cell line, HL60, which overexpresses the c-myc proto-oncogene (Wickstrom E. L., el al., 85 Proc. Natl. Acad. Sci. USA 1028-1032 (1988) and Harel-Bellan, A., el al, 168 Exp. Med. 2309-2318 (1988)).

Inhibitory nucleic acids can also utilize the third approach of designing the “sense” strand of the gene or mRNA to trap or compete for enzymes or binding proteins involved in mRNA translation. Lastly, inhibitory nucleic acids are used to induce chemical inactivation or cleavage of the target genes or mRNA. Chemical inactivation can occur by several mechanisms, for instance, by induction of crosslinks between the inhibitory nucleic acid and the target nucleic acid within the cell. In a particularly preferred embodiment, the expression vector of the present invention includes a sequence of interest that, when transcribed inside the host cell, functions as an enzyme to effect the cleavage of the target nucleic acid.

Focusing now on the expression vector of the present invention, the vector comprises a set of genetic elements adapted for delivery into a cell to produce ssDNA in vitro or in vivo for altering gene expression that includes
(A) an RNA dependent DNA Polymerase (reverse transcriptase) gene, and
(B) a cassette including (1) an inverted tandem repeat (IR), (2) one or mire sequences of interest located (a) between the inverted repeat (IR), (b) 3′ to the IR, or (c) both between the IR and 3′ to the IR and (3) a primer binding site (PBS) for the reverse transcriptase that is located 3′ to the IR as shown in FIG. 2.
   Although not required, the expression system also preferably includes the functions and signaling instructions for transcription of these components in vivo and the functions and signaling instructions for translation of the reverse transcriptase (RT) gene. Additional elements that are optionally included in the expression vector of the present invention may include one or more of an RNAse gene, usually associated with the RT gene, a restriction endonuclease (RE) gene (for a purpose described below), a downstream polyadenylation signal sequence for expression in eukaryotic cells so that the mRNA produced by the sequence of interest includes a poly(A) tail, see FIG. 1),: and a DNA sequence having enzymatic activity when the linear ssDNA folds into the appropriate secondary configuration. Although the present invention is not so limited, in one embodiment of the expression vector, the DNA enzymatic sequence is located within a sequence of interest, regardless of whether the sequence of interest is located between the inverted repeat (IR) or between the 3′ aspect of the IR and the PBS.

As noted above, in a first embodiment of the expression vector described herein, the vector comprises two plasmids, the first of which is adapted for delivering the RNA-dependent DNA polymerase (reverse transcriptase) gene, which preferably also contains an RNAse H gene that is linked with a histidine-proline linker to a restriction endonuclease gene, to the cell. These genes are constructed and inserted into a plasmid vector that contains the necessary transcriptional and translational control elements along with polyadenylation tailing sequences. This plasmid is referred to herein as the “A” plasmid, pssXA, as shown in FIG. 3. A second, “B” plasmid was constructed which, in the embodiment described herein, includes the three above-listed elements of the cassette, namely, a primer binding sequence (PBS) matched to the reverse transcriptase (RT), a sequence of interest (SOI), and an inverted repeat (IR). In this second plasmid, exemplified by the plasmid pssXB shown in FIG. 4, the SOI is located either between the inverted tandem repeats or in a 5′ position (with respect to the mRNA transcript) to the PBS, the PBS being located at the most 3′ aspect of the mRNA transcript, or in both locations. In other words, the SOI is located (1) between the IR, (2) between the IR and the PBS, and/or (3) both between the IR and between the IR and the PBS, and as will be described below, two B plasmids are described herein, one (pssXB-1) with the SOI between the IR (e.g., NotI sites) and the other (pssXB-II) with the SOI between the IR and the PBS (e.g., cloned into the PacI/BamHI sites). Like plasmid A, plasmid B also includes a combination of transcriptional control elements. However, in another preferred embodiment herein, the B plasmid does not include (or require) translational control elements since no protein product is produced from this construct.

In another embodiment described herein, the expression vector of the present invention comprises a single plasmid, shown schematically in FIGS. 5, 6, and 7 and designated as plasmids pssXC, pssXD, and pssXE, respectively, in which the above-described set of genetic elements is incorporated. The components of the B plasmid described above, e.g., the PBS, SOI, and IR, reside in the untranslated 3′ portion of the RT polyprotein in the C plasmid shown in FIG. 5. In other words, when the RT-RNAse H component of the C plasmid is transcribed under control of an appropriate promoter (in the embodiments described herein, the RSV promoter was utilized), the resulting mRNA transcript contains the coding region for the RT-RNAse H polyprotein and, at the end of translation at the stop signals, the additional MRNA transcript contains (3′ to this translated protein) the elements from the B plasmid with further 3′ downstream signaling events for polyadenylation signals, which remain intact from the RT-RNAse H component.

The particular single plasmid expression system described herein does not contain the restriction endonuclease (RE) gene, and therefore does not digest the stem of the stem-loop intermediate formed by the inverted repeats. Consequently, the SOI (including the DNA enzyme) is inserted into either the C, D, or E plasmids only in a 3′ position to the JR, and unwanted vector sequences are removed by premature truncation of the ssDNA product as the transcript encounters the relatively stable stem of the stem:-loop intermediate and is unable to continue transcribing ssDNA from the mRNA transcript. More specifically, as will be made apparent in the following description, each SOI was inserted only within the PacI/BamHI restriction sites of the pssXC and pssXD plasmids.

As will also be apparent from the following description of the B, C, D, E, F. and V plasmids, the plasmids include cloning sites for insertion of the SOI. Both NotI sites (located between the IR) and PacI/BamHI (3′ to the IR, e.g., between the IR and the PBS) sites are provided in the preferred embodiment of the B plasmid described herein. The C and D plasmids described herein include only the PacI/BamHI sites for this purpose. The E, F, and V plasmids include a multiple subcloning site that facilitates subcloning of the SOI. As known in the art, a multiple cloning site (MCS) containing a number of restriction enzyme (usually 4-10) recognition sequences, is designed to make a vector more flexible for the insertion of different DNA sequences. As will be apparent to those skilled in the art, however, only restriction enzymes that do not cut the vector can be chosen. Although many are known; the following is a list of restriction enzymes that can be selected for use in connection with pssXE, pss-XF, pssXV, or any other plasmid constructed in accordance with the teachings of the present invention:

### AfIII, AscI, BsiWI, BsmBI, BspMI, BsrGI, BsBI, ClaI, E10471II, HpaI, NarI, PF1MI, PshAI, SfiI, SgfI, SrfI, Sse8387I, SwaI, XcmI,

as well as the EcoRI, PacI, PstI, and SacII sites that were selected for inclusion in the pssXE vector. Those skilled in the art who have the benefit of this disclosure will recognize that these particular cloning sites were chosen for the particular systems described herein and that other cloning sites may be equally useful for this same purpose. The A plasmid comprising the two plasmid vector system described herein was not intended to include the SOI, but those skilled in the art will also recognize that, if a two plasmid vector system is to be used, the elements of the set of genetic elements of the present invention, and particularly the SOI, may be inserted into either plasmid as may be convenient.

The nucleic acid sequence referred to herein as a cassette provides a template for synthesis of ssDNA in target cells. It is this element that includes the SOI, IR, and PBS. As is the case for the other elements of the set of genetic elements of the present invention, this genetic element is preferably regulated by an appropriate wide spectrum or tissue-specific promoter/enhancer, such as the CMV promoter, or combination of promoters/enhancers, located upstream of the genetic element. Also as is the case for the other genetic elements, the promoter/enhancer can either be constitutive or inducible promoter. As set out in more detail below, those skilled in the art who have the benefit of this disclosure will recognize that a number of other eukaryotic promoters may be used to advantage to control expression of the SOI including SV-40, RSV (non-cell type specific) or tissue-specific glial fibulary acidic protein (GFAP).

The primer binding site (PBS) for initiation of priming for cDNA synthesis is located between the 3′ IR and the polyadenylation signal. The PBS is a sequence that is complementary to a transfer RNA (tRNA) which is resident within the eukaryotic target cell. In the case of the mouse Maloney reverse transcriptase (MoMTLV RT) described herein as being utilized in conjunction with the present invention, the PBS takes advantage of the proline tRNA. The PBS utilized in connection with one embodiment of the present invention was taken from the actual 18 nucleotide sequence region of mouse Moloney virus. Shinnick, T. M., el al., Nucleotide sequence of Moloney murine leukemia virus, 293 Nature 543-548 (1981). In the case of the RT gene from human immunodeficiency virus that was also tested as noted below, the PBS was taken from the nucleotide sequence of HIV. Y. Li, el al., 66 J. Virology 6587-6600 (1992). In short, any PBS that is matched to a particular RT is utilized for this purpose. The PBS is exclusively recognized by a primer tRNA that is endogenous to the target cells. Each tRNA has the ability to recognize a unique sequence (i.e., codon) on the mRNA transcript coding for an amino acid, and has the ability to covalently link to a specific amino acid (i.e., the tRNA becomes “charged” when bound to a specific amino acid). However, a primer tRNA, when bound to the mRNA transcript PBS and not covalently linked with an amino acid (i.e., “uncharged”), may be used to initiate ssDNA synthesis by the RT. For example, the MoMULV RT used in the examples described herein recognizes and uses an uncharged lysine tRNA that in turn recognizes and binds to its unique sequence in the PBS. Thus, each PBS incorporated into the expression system of the present invention must contain the unique sequence recognized by the primer tRNA, and the primer tRNA must be a primer tRNA that is recognized by the particular RT utilized.

Other retroviral RT/RNAse H genes may be used to advantage in connection with the present invention, it being preferred that the RT/RNase H gene be an RT/RNase H gene that is regulated by an appropriate upstream eukaryotic promoter/enhancer such as the CMV or RSV promoter for expression in human cells. RNA-dependent DNA polymerase/RT genes suitable for use in connection with the present invention include those from retroviruses, strains of hepatitis B, hepatitis C, bacterial retron elements, and retrons isolated from various yeast and bacterial species. As found in nature, these RNA-dependent DNA polymerases usually have an associated RNase H component enzyme within the same coding transcript. However, the present invention does not require the naturally-occurring RNase H gene for a particular RT. In other words, those skilled in the art will recognize that various combinations of RT and RNase H genes can be utilized to fulfill this fiinctiorf and that modifications and/or hybrid versions of these enzyme systems are available and/or known to those skilled in the art that function in the intended manner. Those skilled in the art will also recognize that the target cell may itself have sufficient endogenous RNase H to fulfill this function (from, for instance, prior retroviral infection) to fulfill this function. It will also be recognized that the use of a viral vector as the expression vector of the present invention makes possible the use of a number of viral RT genes that are not well-suited for use in a plasmid expression vector system.

The RT/RNase H gene also preferably includes a downstream polyadenylation signal sequence so that the MlRNA produced from the RT/RNase H gene includes a 3′ poly(A) tail for MRNA stability. As known to those skilled in the art, multiple poly(A) tails are available and are routinely used for production of expressed eukaryotic genes.

Those skilled in the art will also recognize that a number of tissue-specific or wide spectrum promoters/enhancers, or combinations of promoters/enhancers other than those listed above may also be used to advantage to regulate the RT/RNAse H gene, the RE gene (if utilized), and the sequence of interest. Although a list of all available promoters/enhancers is not needed to exemplify the invention, as noted above, the promoters/enhancers may be constitutive or inducible and may include the CMV or RSV (non-cell type specific) or GFAP (tissue specific) promoters/enhancers listed here and many other viral or mammalian promoters. Representative promoters/enhancers that are appropriate for use in connection with the cassette of the present invention may include, but are not limited to, HSVtk (S. L. McKnight, et al., 217 Science 316 (1982)), human β-globulin promoter (R. Breathnach, et al., 50 Ann.

Rev. of Biochem. 349 (1981)), β-actin (T. Kawamoto, et al., 8 Mol. Cell Biol. 267 (1988)), rat growth hormone (P.1¢ Larsen, el al., 83 Proc. Natl. Acad. Sci. U.S.A. 8283 (1986)), MMTV (AL. Huang, et al., 27 Cell 245 (1981)), adenovirus 5 E2 (M.J. Imperiale, et al., 4 Mol. Cell. Biol. 875 (1984)), SV40 (P. Angel, et al., 49 Cell 729 (1987)), α-2-macroglobulin (D. Kunz, et al., 17 Nucl. Acids Res. 1121 (1989)), MHC class I gene H-2kb (M. A. Blanar, et al., 8 EMBO J. 1139 (1989)), and thyroid stimulating hormone (V. K. Chattedjee, et al., 86 Proc. Natl. Acad. Sci. U.S.A. 9114 (1989)). A list of other promoters that may be suitable for use in connection with the cassette of the present inventions includes:

1. SV40 early promoter;

2. Cytomegalovirus (CMV) promoter;

3. Elongation factor-1a (EF-1a) promoter;

4. Thyroxine-binding globulin (TBG) promoter;

5. Multidrug resistance gene (mdr1) promoter, drug and heat inducible

6. Heat shock protein (HSP) promoter;

7. Tet-responsive (TRE) promoter, drug inducible;

8. HSV (thimine kinase) TK promoter, heat inducible;

9. Gal4-E1b promoter; drug inducible;

10. Ubiquitin C(UbC) promoter; and

11. Telomerase reverse transcriptase (TERT) promoter, tumor-specific.

Those skilled in the art will recognize that this list of promoters is not intended to be all-inclusive and that there are other promoters that will function to advantage when utilized in the espression vector of the present invention.

The RT produced in the cell synthesizes a complementary DNA (cDNA) using as the template the genetic element including the SOI described below. The RNase H activity of the RT degrades the mRNA template component of the RNA/cDNA hybrid to produce a ssDNA in vivo.

The gene encoding the RE (used in the two plasmid expression system and not a required component of that system) may be any of several genes which encode for REs, and preferably those that are controlled by one or more constitutive or inducible wide spectrum and/or tissue-specific promoters/enhancers such as those listed above. The particular REs tested were MboII and FokI, but those skilled in the art who have the benefit of this disclosure will recognize that any RE (type I, II, IIS, or IR) site may be included in the IR. These enzymes “clip,” or digest, the stem of the stem-loop intermediate described below to linearize the SOI as single-stranded DNA.

Although expression of the RE gene may be regulated by an appropriate constitutive or inducible promoter/enhancer located upstream from the restriction endonuclease gene such as the CMV or RSV promoter for expression-in. human cells, in plasmid pssXA, the RE gene (Mboll) is linked to the RT-RNAse I .polypeptide. The RE gene also preferably incliudes a downstream polyadenylatioh signal sequence so that the mRNA transcript from the RE gene will have a 3′ poly(A) tail.

The cassette of the present invention also comprises an inverted tandem repeat (IR). After digestion of the MRNA from the mRNA-cDNA heteroduplex by RNAse H and the release of the ssDNA, the IR causes the ssDNA to fold back upon itself to form the stem of a stem-loop structure, the stem structure being comprised of double stranded, anti-parallel DNA, in the manner described in U.S. Pat. No. 6,054,299 and as shown in FIG. 2, after the cassette is transcribed in the cell and after the RT/RNase H produced by transcription of the genes produces the ssDNA sequence of interest from the mRNA transcript in the cell. One or more RE site(s), which may be cut by the RE produced from the RE gene (in the case of those plasmids that include an RE gene) or by an endogenous RE, may be designed into the double stranded portion, i.e., the IR, that forms the stem of the stem-loop intermediate. The ssDNA which is produced is transcribed with the encoded 5′ and 3′ regions flanking the stem (made up of the IR) and a loop containing the SOI. The stem is then digested by the RE at the out site designed into the stem (again, note that the endonuclease recognition site may be designed into the stem even though the RE gene is not included in the vector system of the present invention) to release the ssDNA loop (see FIG. 1). The loop portion of the ssDNA, which does not form apparent duplex DNA, is immune to RE activity since REs recognize only double stranded DNA as a target substrate.

As noted above, the RE site(s) need not be designed into the IR which forms the stem of the stem-loop intermediate if it is desired to produce ssDNA from an SOI located between the PBS and the IR with transcription of the cassette terminating at the stem formed by the IR. Another option is to design the IR to contain eukaryotic, prokaryotic, or viral protein DNA binding sites, which can act to competitively titer out selected cellular proteins. Combinations of restriction sites or other sequence specific elements may be included in the IR depending on the base pair composition chosen for the IR such that linear or precisely cut stem-loop intermediate forms of ssDNA are produced. It is generally preferred to use synthetically constructed sequence specific elements in the IR since it is unlikely that a naturally occurring inverted repeat would have the properly aligned restriction sites.

As noted above, the cassette which comprises one of the elements of the set of genetic elements of the present invention may also. include a DNA sequence with catalytic activity. Because of the inclusion of the so-called “DNA enzyme” in the cassette (and in the embodiment described herein, the DNA enzyme is located within the sequence of interest), the present invention is used to particular advantage when the sequence of interest serves as the template for synthesis of an inhibitory nucleic acid that is an antisense sequence or a DNA enzyme sequence. For that reason, the examples set out herein describe production of an antisense SOI as set out in FIG. 5B including a sequence having enzymatic activity against MRNA including a c-raf cleaving enzyme designed specifically to bind to the 3′ untranslated region of the c-raf MRNA, which is targeted by antisense ISIS 5132 (Monia, B. P., et al., 2 Nature Medicine 668-675 (1996), hereby incorporated into the present specification in its entirety by this specific reference). The two 9 bp target specific binding arms were flanked by the 15 bp catalytic domain (Santoro, S. W. and G. F. Joyce, Mechanism and utility of an RNA-cleaving DNA enzyme, 37 Biochemistry 13330-13342 (1998), also incorporated into the present specification in its entirety by this specific reference).

Compatible restriction sites were added to the DNA enzyne oligonucleotides so that they could be inserted into either NotI sites or Pacd and BamHI, and the resulting plasmids were designated as pssXB-I and pss-XB-II, respectively.

Those skilled in the art will recognize that as described above, the expression vector of the present invention is not utilized solely for producing antisense sequences in vivo, that the antisense seque.nce need not necessarily contain a nucleic acid sequence having catalytic activity, and that the nucleic acid sequence could also be 10 any of the other types of inhibitory/excitatory nucleic acid sequences described above.

The above-described SOI was chosen for demonstration of the present invention because the c-raf kinase in A549 lung carcinoma cells system has been well characterized (Monia, et al., supra (1996)). The Raf protein is a serine/threonin protein kinase shown to act as a direct downstream effector of ras protein within the MAP kinase signaling pathway with downstream activiation of MEK1/MEK2 and subsequent activiation of ERK1 and ERK2 (Daum, G., et al., The ins and outs of.iaf kinases, 19 Trends Biol. Sci. 474-480 (1994)). A number of solid tumors and leukemias have been demonstrated to harbor either mutations in ras or have upregulations in MAP kinase signal pathways. Signal transduction pathways such as in c-raf related tumors have been attractive targets for oncological therapies, and the phosphorothioate ODN ISIS 5132 noted above has been demonstrated to be a potent antisense inhibitor (Monia, et al., supra (1996)). Further, ISIS 5132 has been shown to induce apotosis (Lau, Q. C., et al., 16 Oncogene 1899-1902 (1998), also incorporated into the present specification in its entirety by this specific reference) and appears to represent a potential effective treatment against such tumors. This antisense ODN recently entered Phase I clinical trials (O'Dwyer, P. J., et al., C-raf-1 depletion and tumor responses in patients treated with the c-raf-1 antisense oligonucleotide ISIS 5132 (CGP 69846A), 5 Clinical Cancer Res. 3977-3982 (1999)), and may prove to be usefijl in treating c-raf-related tumors. Other SOls that have been cloned into plasmids for expression using the expression system of the present invention include (a) a sequence coding for the partial sequence of the 23^{rd} codon of h-ras antisense binding sequence with the 10-23 DNA enzyme sequence (Santoro and Joyce, supra (1997)) inserted between the 5′ and 3′ complimentary sequences, (b) a sequence coding for the partial sequence of pleiotropin antisense binding sequence with the 10-23 DNA enzyme sequence inserted between the 5′ and 3′ complimentary sequences, (c) a sequnce coding for the partial sequence of tat antisense binding region of the SIV sequence with the 10-23 DNA enzyme sequence inserted between the 5′ and 3′ complimentary sequences, (d) a sequence coding for a guanine-rich, triplex-forming, 30 nucleic acid long ODN with high affinity for third-strand binding in anti-parallel triplex fashion to a 30 bp G-rich polypurine sequence that was inserted into the region between the two mutant thymidine kinase (TK) genes in the mouse fibroblast cell line FL-10 designated AG30 (Luo, Z., et al., High-frequency intrachromosomal gene conversion induced by triplex-forming oligonucleotides microinjected into mouse cells. 97 Proc. Natl. Acad. Sci. USA 9003-9008 (2000)), and (e) a sequence coding for the B-gal reporter gene including a DNA enzyme sequence.

The nucleic acid sequence having enzymatic activity utilized in altering gene expression is the 10-23 DNA enzyme (Santoro and Joyce, supra (1997)). The enzymatic sequence is inserted into the cassette in either or both of the two locations, e.g., (a) between the IR and inside the SOI (at the NotI site) or (b) inside the second SOI that is located 3′ to the IR and 5′ to the PBS (at the PacI/BamHI sites). Either way, the resulting ssDNA is specific for the target DNA sequence(s), mRNA sequence(s), or any other suitable substrate, to inhibit or change DNA or mRNA splicing mechanisms, or even to directly alter the cellular genome in a specific manner.

Those skilled in the art will recognize from this disclosure that any DNA sequence having enzymatic activity will function for the intended purpose when inserted into the cassette of the present invention. A number of nucleic acid sequences with enzymatic activity have been reported in the literature, including:
sequences having RNAse activity such as the so-called “10-23” and “8-17” enzymes (Santoro, S. W. and G. F. Joyce, siepra (1997)) and other metal-dependent RNAses (Breaker, R. R. and G. F. Joyce, 1 Biol. Chem. 223-229 (1994) and Breaker, R. R. and G. F. Joyce, 2 Biol. Chem. 655-660 (1995)) and histidine-dependent RNAse (Roth, A. and R. R. Breaker, 95 Proc. Natl Acad. Sci. USA 6027-6031 (1998));
sequences having DNAse activity such as copper-dependent DNAse (Carni, N., et al., 3 Chem. Biol. 1039-1046 (1996), Carni, et al., supra (1997); Sen, D. and C. R. Geyer, 2 Curr. Opin. Chem. Biol. 680-687 (1998)) and the DNAses which required divalent metal ions as cofactors or hydrolyzed the substrate independently of divalent metal ions reported in Faulhammer, D. and M. Famulok (269 J. Molec. Bio. 18-203 (1997));
sequences with DNA ligase activity such as copper-dependent DNAse (Breaker, R. R., 97 Chem. Rev. 371-390 (1997)) and zinc-dependent E47 ligase (Cuenoud, B. and J. W. Szostak, 375 Nature 611613 (1995));

sequences with DNA kinase acitivity such as calcium-dependent DNA kinase (Li, Y. and R. R. Breaker, 96 Proc. Natl. Acad. Sci. USA 2746-2751 (1999)); and
sequences with RNA kinase acitivity such as calcium-dependent DNA kinase (Li, Y., sipra (1999)).
   Generally, it is those DNA sequences having enzymatic activity that are derived from physiological conditions that are preferred for use in connection with the cassette of the present invention.

It is preferred that the expression vector of the present invention contain other specialized genetic elements to facilitate the identification of cells that carry the vector and cassette and/or to increase expression of the genetic elements comprising the cassette. The specialized genetic elements include selectable marker genes so that the vector can be transformed and amplified in a prokaryotic system. For example, one of the SOI's included in an expression vector constructed in accordance with the teachings of the present invention is a sequence encoding the AG30 TFO. When transfected into parental mouse LTK parental cells that lack TK, recombination between the two TK genes induced by the AG30 TFO molecule has the potential to generate wild-type TK and can be selected by growth in the presence of HAT medium. Other commonly uised selectable markers are genes that confer to the bacteria (e.g., E. coli) resistance to antibiotics such as ampicillin, chloramphenicol, kanamycin (neomycin), or tetracycline. It is also preferred that the vector contain specialized genetic elements for subsequent trarlsfection, identification and expression in eukaryotic systems. For expression in eukaryotic cells, multiple selection strategies (e.g., Chinese Hamster Ovarian: CHO) may be used to confer resistance to an antibiotic or other drug. These strategies may be used to alter the phenotype of the cell with results such as morphological changes, loss of contact inhibition, or increased growth rate. Selectable markers used in eukaryotic systems include, but are not limited to, resistance markers for Zeocin, resistance to G418, resistance to aminoglycoside antibiotics, or phenotypic selection markers.

Incorporation of these components into the expression vector of the present invention makes available at least two convenient methods for removing predetermined vector sequences amer the production of ssDNA as shown in FIG. 1. In the first, the cassette comprising the expression vector is reverse transcribed in the host cell from the PBS so that the SOI between the IR comprises the loop portion of the ssDNA stem-loop intermediate produced when the nucleotides comprising the IR pair up to form the stem o0L the stem-loop vector, the stem comprising an RE site. After digestion with the appropriate RE, the loop is released as linearized, single-stranded cDNA without (and/or with minimal) flanking sequences. In the second method, the cassette is reverse transcribed from the PBS and an SOI included in the cassette 3′ to the IR is likewise transcribed, but reverse transcription is terminated at the stem of the stem-loop structure formed by the pairing of the nucleotides of the IR. Either way, the resulting ssDNA is produced with minimal flanking sequences. If it is desired to produce ssDNA utilizing the second method, the cassette is designed with an IR that forms a stem that is more stable than the stem produced when ssDNA is produced by digestion of the stem in accordance with the first aspect of the present invention (for instance, by designing the IR so as not to include an RE site). By designing the cassette with an IR that forms a stem that is easily denatured in accordance with the first aspect of the invention, reverse transcription proceeds right on through the second SOI (if it is even designed into the cassette) to the SOI located between the IR. This “premature termination” of the reverse transcriptase cDNA transcript at the 3′ aspect of the stem structure therefore provides a second method for limiting the intervening vector sequences contained with an in vivo-produced ssDNA. A stem that is intermediate in stability allows production of both the first and second SOIs.

It will also be evident to those skilled in the art from this description that the intact stem-loop ssDNA structure can function similarly in many applications as the linearized ssDNA form. Consequently, the cassette is also used to advantage without the restriction endonuclease gene and associated regulatory elements and/or with a sequence of interest which lacks the corresponding restriction endonuclease site.

It will also be evident to those skilled in the art from this description that a cassette can be made which encodes a ssDNA that has a “trimmed” stem-loop structure. The RE sites encoded in the IR flanking the SOI are designed such that the stem portion (after duplex formation) is digested with the corresponding RE so as to cut the dsDNA comprising the stem in a way that removes a portion of the stem and the associated flanking sequences, yet leaves sufficient duplex DNA that the transcript retains the stem-loop structure. Such a ssDNA structure may be more resistant to intracellular nucleases by retaining the “ends” of a ssDNA in double stranded form.

The expression vector of the present invention is delivered to the target cell by multiple delivery routes depending upon the particular target cell. For example, viral vectors are frequently used for introducing DNA into the genome of a target cell. In an indirect method, viral vectors are used to infect target cells removed from the body and the infected cells are then re-implanted (i.e., ex viio). Direct in vivo gene transfer into postnatal animals has been reported for formulations of DNA encapsulated in liposomes and DNA entrapped in proteoliposomes containing viral envelope receptor proteins. Nicolau, et al., 80 Proc. Natl. Acad Sci USA 1068-1072 (1983); Kaneda, et al., 243 Science 375-378 (1989); Mannino, et al., 6 Biotechniques 682-690 (1988). Positive results have been obtained with calcium phosphate co-precipitated DNA. Benvenisty and Reshef, 83 Proc. Natl. Acad. Sci. USA 9551-9555 (1986). Other systems used to advantage to administer the expression vector of the present invention include intravenous, intramuscular, and subcutaneous injection, as well as direct intra-tumoral and intra-cavitary injection. The expression vector of the present invention is also conjugated to specific antibodies for delivery to a target host cell or packaged in liposomes having binding characteristics enabling the liposome to target a specific cell or tissue. The expression vector of the present invention is also administered through topical, transmucosal, rectal, oral, or inhalation-type methods of delivery.

The expression vector of the present invention is utilized to deliver antisense, triplex, or any other inhibitory nucleic acid, excitatory nucleic acid, or single-stranded nucleotide using known digestion and ligation techniques to splice the particular SOI into the vector (between inverted tandem repeats or between PBS and inverted tandem repeats). Those skilled in the art who have the benefit of this disclosure will also recognize that the above-described signals used for expression within eukaryotic cells may be modified in ways known in the art depending upon the particular host cell and sequence being targeted. For. instance, a likely modification is to utilize a promoter that confers advantageous expression characteristics on the system in which the SOI is to be expressed. As noted above, there are many promoters that are used to advantage with the expression vector of the present invention; indeed, there are so many possible promoters (and other signals), and they are so dependent on the particular target cell for which the sequence of interest has been selected, that it is impossible to list all the potential enhancers, inducible and constitutive promoter systems, and/or poly(A) tailing systems that may be preferred for a particular target cell and SOI.

In one embodiment, the present invention takes the form of a kit comprised of a plasmid into which the above-described RNA-dependent DNA polymerase gene(s) is cloned, having the multiple cloning site (MCS) described in connection with the E plasmid into which the user of the kit inserts a particular SOI. The kit preferably also includes the ligases and other enzymes, along with suitable buffers, for ligating the SOI into the plasmid, a map of the plasmid, and may also include the RE(s) for the MCS into which the SOI is to be cloned.

In the specific embodiments described herein, the SOI(s) is/are delivered to a host cell either by co-transfection of the cells with two plasmids, designated A and B, each plasmid being designed and constructed to include the components listed above, or by a single C, D, or E plasmid. In the two plasmid system, the B plasmid encodes the cassette including the SOI, either nested wvithin flanking sequences that include the IR or between the IR and the PBS that provides the post-transcriptional processing signals that mediate the conversion of the MRNA into ssDNA. Activities required for processing the primary gene product of the B plasmid into ssDNA, with the removal of vector sequences and processing signals, specifically the RT/RNAse H, and RE (if utilized), are expressed from the A plasmid. The single-stranded DNA sequence that is released by interaction of the transcriptional products of these components in vivo is free to bind intracellular targets such as mRNA species and DNA promoters in antisense, DNA enzyme and triplex strategies.

As noted above, the B plesmid includes cloning sites (NotI sites were utilized in the B plasmid described herein) between which any DNA SOI is placed (in the examples described herein, the SOI is an antisense sequence to c-raf kinase including the 10-23 enzyme sequence, but as described above, other sequences that have been produced in vivo using the plasmids described herein include a “stuffer,” or test, sequence, telomeric repeats, h-ras, a region encoding the angiogenic growth factor pleiotrophin, the region encoding tat (from SIV), the AG30 TFO, and a sequence that targets the β-gal protein translation start site). Flanking the cloning-sites are signals directing the processing of the primary rnRNA transcript, produced from a promoter (a CMV promoter was utilized in the B plasmid described herein), into the desired single-stranded inhibitory nucleic acid. After cloning the desired SOI into the B plasmid, the A and B plasmids are co-transfected into a cell line of choice for constitutive expression of ssDNA. Similarly, in the single plasmid expression system described herein, the SOI is cloned into that plasmid and transfected into the cell line for further processing. Regardless of the distribution of the elements of the above-described set of genetic elements between two (or even more) plasmids, or if the elements are all contained ini a single plasmid, this processing proceeds in three steps following transcription of the single-stranded DNA region (i.e., .SOI, IR, and PBS):
(1) reverse transcription of the plasmid RNA transcript by RT, which in the embodiments described herein is an RT expressed by the A, C, D, or E plasmid (in the embodiment described herein, the RT is MoMuLV RT), proceeding from the primer binding site lying 3′ to the SOI (the SOI optionally including the sequence with enzymatic activity), IR, and PBS;
(2) RNAse H digestion of the resulting heteroduplex, either by RNAse H activity of the RT polyprotein or by endogenous RNAse H activity, to release the single-stranded DNA precursor from its RNA complement; and
(3) Removal of flanking sequences by either digestion of the stem of a stem-loop intermediate formed upon Watson-Crick base pairing of the bases comprising the IR or by premature termination of the cDNA transcript by formation of the stem-loop secondary structure by the self-complementary IR.
   Those skilled in the art will recognize that the particular cloning sites flanking the SOI, the particular RT, RE (if utilized), promoter, PBS, and all the other elements of the expression vector of the present invention, are chosen depending upon the particular SOI and/or system in which the ssDNA is to be expressed.

### EXAMPLES

Except where otherwise indicated, standard techniques as described by Seabrook, et al. (1989) (J. Seabrook, et al., Molecular Cloning: A Laboratory Manual (2nd Ed.), Cold Spring Harbor Press (1989), hereinafter referred to as “Maniatis, et al. (1989)”) and Ausubel, et al. (1987) (F. M. Ausubel, et al., Current Protocols in Molecular Biology, New York: John Wiley & Sons (1987)), both of which are hereby incorporated in their entirety by this specific reference, were utilized in the examples set out below. It should be understood that other methods of production of ssDNA, both by natural processes and by methods using different enzyme products or systems, may also be utilized in connection with the method of the present invention and that the examples set out herein are set out for purposes of exemplification and are not intended to limit the scope of this Disclosure or the invention described herein.

The plasmid pcDNA3.1Zeo+ was purchased from Invitrogen Corp. (Carlsbad, Calif.) and plasmid pBK-RSV from Statagene (La Jolla, Calif.). Oligodeoxynucleotides (ODN) were synthesized by Midland Certified Reagent Co. (Midland, Tex.). Polymerase chain reactions (PCR) were carried out using Taq DNA polymerase purchased from Boehringer Mannheim Corp. (Indianapolis, IN) in a Robo-gradient thermal cycler (Stratagene (La Jolla, Calif.)). Restriction endonucleases and T4 DNA ligase were obtained from Boehringer Mannheim Corp. (Indianapolis, Id.). The ODNs used are listed in the attached Sequence Listing.

All ODNs were allowed to hybridize in 1 μl (5 μg/μl in water) in separate tubes which were incubated at 70° C. for 5 min and allowed to hybridize for 15 min at room temperature. Standard restriction endonuclease digests were carried out (EcoRI used as a negative control) w.ith 10 units of enzyme in a total reaction volume of 15 pl and appropriate reaction buffers. DNA fragments were resolved in and isolated from agarose gels. The selection of positive clones on ampicillin plates was performed after transformation into competent XL1-Blue MRF cells (Stratagene) as described by Maniatis, et al. (1989). After positive clones were selected, plasmid DNA was isolated using the above-described Quiagen plasmid isolation kit.

Construction of plasmids. The construction of six expression plasmids is described. The first, pssXB (FIG. 3), was derived from pcDNA3.1Zeo(+) (Invitrogen Corp.) and contains the genetic element encoding the ssDNA sequence of interest. pcDNA3.1Zeo(+) was digested with restriction endonucleases HindIII and NotI at positions 911 and 978, respectively. The double-stranded linker region having compatible HindIII and NotI ends formed by annealing the synthetic, single stranded oligodeoxynucleotides ODN-5′-N/M(link)2-H/N and ODN-3′-N/M(link)2-H/N was ligated under standard conditions into the HindIII/NotI double-digested pcDNA3.1Zeo(+) transformed into Surell cells (Stratagene, Inc.). The ODNs were allowed to hybridize in 1 μl (5 μg/μl in water) in Ependorf tubes incubated at 70° C. for 5 minutes and allowed to hybridize for 15 minutes at room temperature. Appropriate clones were selected and sequenced to assure proper insertion of the linker region. The resulting plasmid was termed pssXB. pssXB is shown in FIG. 4A and is the plasmnid into which the sequence of interest (FIG. 4B) is cloned. For cloning sequences of interest between the inverted tandem repeats, the two NotI sites at positions 935 and 978, respectively (see FIG. 4A), were used. These two sites are contained within the inverted tandem repeats. For inserting sequences of interest between the inverted tandem repeats and the primer binding site, two convenient restriction endonuclease sites, PacI and BamHI, at positions 1004 and 1021, respectively, were used.

The second plasmid, pssXA (FIG. 3), is also a component of the two plasmid vector system. The “A” plasmid contains the Mo-MuLV-RT (Shinnick, T. M., et al., 293 Nature 543-548 (1981)) and restriction endonuclease genes and was derived from pBK-RSV (Stratagene), also using XL-1 Blue MRF′ as the host cell. A mouse cell line expressing Moloney murine leukemia virus was obtained from the American Type Culture Collection (#CRL-1858). Viral RNA was isolated from cells in accordance with the method described in Chomczymski, P. and N. Sacchi (162 Anal. Biochem. 156-159 (1987)) using Trizol reagent (GibcoBRL) and reverse transcribed using primer 3′-RT-HindIII (5′-CTTGTGCACAAGCTTTGCA-GGTCT-3′). The transcript was then PCR amplified using the TaqPlus long polymerase system (Stratagene) for 35 cycles: 94° C. 1 min, 67° C., 1 min, and 72° C., 2.5 min. Primers used for the PCR reaction were 5′-RT-SacI (5′-GGGATCAGGAGCTC-AGATCATGGGACCAATGG-3′) and 3′-RT-HindIII, same as used for reverse transcription, and include compatible SacI and HindIII sites, respectively. The 2.4kb product obtained included the sequence of the Mo-MuLV between positions 2546 and 4908. The mature viral RT peptide is encoded by the sequence between positions 2337 and 4349 (Petropoulos, C. J., Retroviral taxonomy, protein structure, sequences and genetic maps, in J. M. Coffin,et al.(Eds.), Retroviruses, New York: Cold Spring Harbor Laboratory Press, pp. 757-805 (1997)), but the peptide truncated at the amino terminus retains full activity (Tanese, N. and S. P. Goff, 85 Proc. Natl. Acad. Sci. U.S.A. 1777-1781 (1988)). The peptide encoded by this construct includes part of the integrase gene, which follows the RT in the MoMuLV polyprotein (Petropoulos, supra).

The bacterium Moraxella bovis, which encodes the restriction endonuclease MboII (Bocklage, H., et al., 19 Nucleic Acids Res. 1007-1013 (1991)), was, obtained from the American Type Culture Collection (ATCC#10900). Genomic DNA was isolated from M. bovis using the Stratagene DNA extraction kit following the manufacturer's instructions and used as the template DNA in the PCR. Using two primers, 5′-MboII-HindIII (5′-CAATTAAGGAAAGCTTTGAAAAATTATGTC-3′) and 3′-MboII-Xmal (5′-TAATGGCCCGGGCATAGTCGGGTAGGG-3′), the MboII gene was PCR amplified from genomic DNA for 30 cycles: 94° C., 30 sec., 58° C., 1 min., 72° C., 1 min. These primers were designed to include a HindIII and an XmaI site, respectively. The 1.2 kb product, copying the M. bovis genome between positions 888 and 2206, contains the coding region for the MboII enyyme.

The pBK-RSV vector was digested with XmaI and NheI. The NheI end was converted to a SacI end using linker formed by two annealed oligonucleotides, 5′-Nhe-Sac-link (5′-CTAGCGGCAAGCGTAGCT-3′) and 3′-Nhe-Sac-link (5′-ACGCTTGCCG-3′). The RT and MboII amplimers were ligated through the HindIII site and the construct was subsequently ligated between the Sacd and XmaI sites of pBK-RSV to give pBK-RSV-RT/MboII.

To insert a flexible linker between the RT and MboII domains of the polyprotein, a fragment of pBK-RSV-RT/MboII plasmid lying between the AseI and Bg/II sites, which encodes the 5′ end of the MboII gene and part of the integrase gene, was excised and replaced with an insert containing a 6-His-linker and 5′-MboII DNA fragment deleted by the double digestion. The insert was obtained by mutually-primed DNA synthesis from two templates, Rep(+) (5′-ATACTATTAATTTTGGCAAATCA-TAGCGGTTATGCTGACTCAGGTGAATGCCGCGATAATTTTCAGATTGCAAT CTTTCATCAATGAATTTCAGTGATGAAT-TGCCAAGATTGATGTTGC-3 ) and Rep(−) (5′-GACGAGATC-TCCTCCAGGAATTCTCGAGAATTCGGATCCCCCGC-TCCCCACCACCACCACCACCACCCTGCCCCGCGGATGAAAAATTATGTGAG CAACATCAATCTTGGC-3′), that have complementary sequences of 17 bases at the 3′-ends. These two oligonucleotides were annealed and extended with the modified T7 DNA polymerase (USB) and the double-stranded oligonucleotide was then digested with AseI and BglII and inserted into the pBK-RSV vector to give pssXA (FIG. 3).

In a third embodiment of an expression vector constructed in accordance with the teachings of the present invention, the pc3.1DNAZeo(+)-derived “B” plasmid and the pBK-RSV-derived “A” plasmid were fused such that resulting plasmid encoded all of the elements of the present invention, including the ssDNA-encoding sequence of interest, the tandem inverted repeat, and the Mo-MuLV-RT gene. To produce this “C” plasmid, plasmid pssDNA-Express-A was digested with SacI XmaI to remove the MboII gene. A linker region comprised of oligonucleotides 5′-(link)2-Hind/Xba (5′-CCGGATCTAGACCGCAAG-CTTCACCGC-3∝) and 3′-(link)2-Hind/Xba (5′-GGTGAAGCTTGCGGTCTAGAT-3′), which were allowed to anneal at 70° C. for 15 minutes and slowly cooled to room temperature, was ligated into the plasmid after digestion under standard conditions. Positive clones were harvested and sequenced to verify linker placement and this plasmid was then digested with Xba and HindIII. The plasmid pssDNA-Express-B was then digested with HindII and Xba and the corresponding 300 base pair DNA fragement containing the previously described inverted tandem repeats, multiple cloning site, and PBS was cloned into the digested plasmid to give pssXC (Fig. 5A). Standard ligation reactions were performed and transformed into Sure II cells (Stratagene, Inc.). Transformed positive colonies were harvested and positive clones were identified by restriction analysis.

The sequences of interest were cloned into pssXC using the BamHI and PacI sites in the multiple cloning site (Fig. SB). Four different SOIs were synthesized for these constructs as described above, and similar procedures were utilized for inserting each SOI. Each construct was prepared by allowing the paired ODNs to anneal at 70° C. for 15 minutes and cooling to room temperature, followed by ligation into the plasmid under standard conditions. After transformation into SureII cells, appropriate colonies were selected with verification by sequencing for the individual inserts.

A third expression vector constructed in accordance with the present invention, pssXD, was prepared by combining the pssXA and pssXB plasmids in the following manner. pssXA, which contains the Mo-MuLV reverse transcriptase (RT), was digested with XmaI and BglII and the resulting XmaI-BglII fragment was replaced with a double-stranded DNA adaptor formed by annealing two oligos, XmaI-BgIII-Stop 1 (5′-CCGGATCTAGACCGCAAGCTTCATTTAAA-3′) and XmaI-BgIII-Stop 2 (GATCTTTAAATGAAGCTTGCGGTCTCGAT-3′). This adaptor contains a protein translation stop codon and subcloning sites, XbaI and HindII, the stop codon being used to terminate RT protein translation. The resulting plasmid was designated pssXD (FIG. 6A). XbaI-HindIII fragments were cleaved from both pssXB and pssXB-II and then cloned into pssXD between XbaI and HindIII. These DNA fragments contain: 1) RT primer binding site (PBS); 2) stem-loop structure; and 3) random control sequence (pssXB) or c-raf DNA enzyme sequence (pssXB-II). The resulting plasmids were designated pssXD-I and pssXD-II, respectively. A RSV promoter regulates gene expression of all elements necessary for ssDNA expression and all elements are transcribed as a single mRNA molecule. Endogenous tRNA^{pro} binds to the PBS on the 3′ end of the transcript, and is used as the primer for single-stranded DNA synthesis (Marquet, et al., 77 Biochimie 113-124 (1995)). After reverse transcription of the single-stranded DNA by RT, the ssDNA is released when the template mRNA is degraded either by endogenous RNase H or the RNase H activity of the RT (Tanase and Goff, 85 Proc. Natl. Acad. Sci. U.S.A. 1777-1781 (1988)).

A fourth expression vector constructed in accordance with the teachings of the present invention is the pssXE plasmid shown in FIG. 8. To make pssXE, the pssXB and pssXD plasmids were double-digested with NheI and XhoI. The DNA fragment digested from pssXD (containing RT, PBS, and other component necessary for synthesis of ssDBA) was subdloned into the vector digested from pssXB that contained a CMV promoter. A multiple coloning site (MCS) was created in order to facilitate subcloning the sequence of interest into the plasmid. 5′-E/S/P/P-LINKER (5′-TCGAGCGGCCAGGGGTCTCCCGATCCCGGACGAGCCCCCAAAGAATTCCG-CGGCTGCAGTTAAT-3′) and 3′-E/S/P/P-LINKER (5′-TAACTGCAGCCGCG-GAATTCTTTGGGGGCTCGTCCGGGATCGGGAGACCCCTGGCCGC-3′) were annealed and subcloned into PacI and XhoI sites of pssXD plasmid to create four new restriction enzyme recognition sites, EcoRI, SacII, PstI and PacI.

A fifth expression vector, pssXF, pssXF was constructed by inserting a double-stranded oligo, E/S/P/P, into the PacI and XhoI sites of pssXD and is shown in FIG. 9. Constructed using the same techniques as described in the preceding paragraphs, this vector includes: a) RSV promoter; b) mouse moloney leukemia viral reverse transcriptase gene; c) primer binding site (PBS); d) multiple cloning site, EcoRI, SacII, PstI and PacI; e) stem-loop structure; and f) neomycin gene.

A sixth expression vector, pssXV (FIG. 10), was constructed by subcloning an NheI+XhoI DNA fragment, digested and isolated from pssXE, into pVAX (Invitrogen Inc.). The pssXV vector includes: a) CMV promoter; b) mouse moloney leukemia viral reverse transcriptase gene; c) primer binding site (PBS); d) multiple cloning site; e) stem-loop structure; and e) Kanamycin gene.

A seventh expression vector (not shown), is prepared from pHi-2-MCS (obtained from the University of Arizona) by inserting a double-stranded oligo (the double-stranded oligo was prepared by annealing two oligos, 5′NheEcoRPacXhoNot, 5′-CTAGCGAATTCTTAATTAACTCGAGGT-3′ and 3′NheEcoRPacXhoNot, 5′-GGCCACCTCGAGTTAATTAAGAATTCG-3′) into the Nhel and NoIl sites. The NheI+XhoI DNA fragment digested and isolated from pssXE (FIG. 7) is then subcloned into the modified pHi-2-MCS. The new construct was designated pssXH and includes: a) HIV2 and CMV promoters; b) mouse moloney leukemia viral reverse transcriptase gene; c) primer binding site (PBS); d) multiple cloning site, EcoRI, SacII, PstI and Pacl; e) stem-loop structure; and f) Neomycin and ampicillin genes.

Tissue culture studies. Stable and transient transfections were carried out by using DOTAP liposomal transfection reagent (Boehringer Mannhiem Corp., Indianapolis, Ind.) using the manufacturer's instructions. All plasmids were transfected into A549 lung carcinoma cell line (ATCC CCL-185) and HeLa cell lines maintained in Dulbecco's Modified Eagles Medium (DMEM) supplemented with 10% Fetal Bovine Serum (FCS) (GibcoBRL, Gaithersburg, Md.). ssDNA assays were performed by PCR and by dot-blot analyses 2448 hours after transfection. ssDNA was isolated from cells transfected 48-72-hr earlier. The ssDNA, which co-localizes with total RNA (Mitrochnitchenko, O., et al., Production of single-stranded DNA in mammalian cells by use of a bacterial retron, 269 J. Biol. Chem. 2380-2383 (1994)), was carried out using Trizol reagent (Gibco Life Technologies, Gaithersburg, Md.). Assays for specific ssDNA species were carried out by both PCR based assays for internal fragment and by denatured single stranded gel electrophoresis with subsequent nylon blotting and probing with an internal biotin-labeled probe.

In more detail, reverse transcniptase activity was assayed using the RT-PCR assay developed by Silver, J., et al. (An RT-PCR assay for the enzyme activity of reverse transcriptase capable of detecting single virions, 21 Nucleic Acids Res. 3593-3594 (1993)), with modifications as set out below. pssXA transfected cells were lysed with lysis buffer (1% Triton™, 1 mM MgCl₂, 100 mM NaCl, 10 mM TRIS-HCl, pH 8.0 and 2 nM DTT), centrifuged at 18,000g for 30 min., and the supernatant collected and frozen at −80° C. until use. Brome mosaic virus (BMV) RNA, used as a template, was reverse transcribed by incubation with the lysate, which would contain RT activity, for 10-30 min. at 37° C. Using primers 5′-CGTGGTTGACACGCAGACCTCTTAC-3′ and 5′-TCAACACTGTACGGCACCCGCATTC-3′, the reverse transcription product was PCR amplified for 40 cycles: 94° C., 20 sec., 56° C., 20 sec., and 72° C., 20 sec. RT-PCR products were analysed by 1.5% agarose gel as shown in FIG. 6.

This RT-PCR assay relies upon RT activity in the cell lysates of transfected cells to produce a cDNA transcript of the BMV RNA substrate. The replication cycle of this virus does not involve a DNA intermediate, eliminating the possibility that an amplification product could be produced without prior reverse transcription. RT activity was determined in the lysltes of A549 cells transfected with the pssXA plasmid (lanes 3 and 4) and the E10 clone, which showed relatively high expression (lanes 5 and 6). RT activity was also determined from A549 cells transiently transfected with control pBK-RSV plasmid (lanes 1 and 2). For transient transfection, lysates were prepared 48 hours after transfection. Results show that cell lysates from both transient and stable transfected (E10) cells support the production of a band of expected size, 150 bp (lanes 3-6), whereas control lysates showed none (lanes 1 and 2).

To detect ssDNA expressed in mammalian cells by pssXB-I and pssXB-II when co-transfected with pssXA into A549 cells (E10), a PCR reaction was carried out using T7 primer and c-raf DNA enzyme specific primer 5′-CTAGCTACAACGAGACATGC-3′. Total RNA fraction was used as template and pre-treated with either S1 nuclease or RNAse A for 30 min. at 37° C. or left untreated. The pre-treated RNA samples were then PCR amplified for 30 cycles: 94° C., 45 sec., 55° C., 45 sec., and 72° C., 30 sec. PCR products were analyzed by 8% acylamide-gel as shown in FIG. 7 (lanes 1 and 3, S1 nuclease; lanes 2, 4, and 5, RNAse). A band of the expected size was produced from both treated total RNA preparations (lanes 2 and 4) and untreated preparations (data not shown). Control preparations treated with S1 nuclease, a highly specific, ssDNA endonuclease, resulted in no amplified products (lanes 1 and 3).

The existence of c-raf DNA enzymes was confirmed by dot-blot detection of ssDNA, using the North2South Chemilurninescent Nucleic Acid Hybridization and Detection Kit (Pierce) following the manufacturer's instructions. Two μg of total RNA, isolated from cells transfected with either pssXA/pssXB-I or pssXA/pssXB-II, or pssXA or untransfected cells, was used. The sequence of c-raf specific, biotin-labeled probe is 5′-GGCCGCACTAATGCATGTCTCGTTGTAGCTAGCCCAGG-CGGGAAGTGC-3′. As shown in FIG. 8, a biotin-labeld c-raf specific oligo probe can only detect signal in the RNA preparations isolated from E10 cells transfected with pssXB-I or pssXB-II but nod, untransfected E10 cells or A549 cells.

To determine whether single-stranded c-raf DNA enzyme expressed with the pssXA/pssXB expression vector altered c-raf MRNA expression, northern blot analysis was performed. The EIO cell line was transiently transfected with either pssXB-I or pssXB-II. At 24 and 48 hrs, cells were harvested for total RNA preparation. Fifteen μ g of total RNA was separated on denatured agarose gel for Northern blot analysis. After overnight transfer, membrane was fixed and probed with both ³²P-labeled c-raf DNA fragment and glyceraldehyde-3-phosphate dehydrogenase (G3PDH), a housekeeping gene. Using random-primed labeling kit from Boehringer Mannheim, c-raf probe was prepared from an IMAGE™ cDNA clone (ID 645539, Research Genetics), that includes a coding region of c-raf kinase gene from position 571 to 2028. G3PDH was also ³²P-labeled and used for normalization of the RNA blot. The membrane was washed with 2×SSC, 0.1% SDS for 15 min. and 0.1 ×SSC for 5 min. The blot was then exposed to X-ray film or quantitated by Molecular Dynamics Phopholmager™. The quantitation result of a representative experiment by phosphor-imaging is shown in graphical form in Fig. 11. Compared to controls transfected with pssXB containing unrelated sequences, pssXB-II reduces c-raf mRNA level to 81% in 24 hrs and 66% in 48 hrs. pssXB-I had a similar effect, reducing c-raf mRNA level by 35% after 48 hrs incubation. It was also observed that there was significantly more cell death (approximately by a third) in the cells transfected with pssXA/pssXB vector expressing c-raf DNA enzyme compared to the control. Only remaining adherent cells were harvested, and not those that began to “float,” so the degree of MRNA reduction may be greater than the 34-36% reduction measured.

The single plasmid expression vector pssXC was transfected into HeLa cell lines. Assays for ssDNA were performed by PCR and by dot-blot analyses 24-48 hours after transfection as described above. Reverse transcriptase activity was assayed using the Silver, et al. (1993) RT-PCR assay described above. Individual colony isolates of stably substituted HeLt cell lines (A12 and B12) were additionally assayed for RT activity. The ssDNA was isolated from cells transfected 48-72-hr earlier. The ssDNA, which co-localizes with RNA, was carried out using Trizol reagent (Gibco Life Technologies, Gaithersburg, Md.). Assays for specific ssDNA species were carried out by both PCR based assays for internal fragment and by denatured single stranded gel electrophoresis with subsequent nylon blotting and probing with an internal biotin-labeled probe. This experiment showed that human tissue culture cells (HeLa cell line), transfected with plasmids designed to synthesize a processed ssDNA, produced ssDNA of the predicted size. As described in the above-incorporated application Serial No. 09/397,782, the ssDNA sequence of interest produced in vivo from pssXC is produced from either the position between the inverted repeats after digestion of the stem of the stem-loop intermediate or from the position between the inverted repeats and the primer binding site by premature termination of the reverse transcriptase cDNA transcript at the 3′ aspect of the stem structure.

Using the total RNA fraction, the expression of intracellular single-stranded c-raf DNA enzyme was determined by a simple dot-blot analysis. The biotin-labeled c-raf specific oligonucleotiee probe used was synthesized by Intergrated DNA Technologies (Coralville, Iowa), and was used to detect signals in the RNA samples isolated from A549 cells either transfected with control pssXD-I or pssXD-ll containing the c-raf DNA enzyme sequence. Two jg of total RNA were pretreated with RNase A to rule out non-specific hybridization to RNA, and in the presence and absence of S1 nuclease for 30 min at 37° C. Subsequently, samples were loaded onto a Hybond-N+ membrane (Amersham Pharmacia Biotec, Piscataway, N.J.), and fixed by UV exposure for 3 min. Hybridization and signal detection were performed using the North2South Chemilurninescent Nucleic Acid Hybridization and Detection Kit (Pierce, Rockford, Ill.). FIG. 14 shows that only cells transfected with pssXD-II displayed a positive signal and that in the presence of S1 nuclease, no detectable signal was observed due to the specific degradation of ssDNA enzyme by S1 nuclease.

To determine whether single-stranded DNA enzyme expressed in A549 cells altered c-raf mRNA levels, quantitative RT-PCR was conducted. c-raf mRNA was quantitated by RT-PCR as described by Li, el al. (7 Gene Therapy 321-328 (2000)) with some modification. Briefly, one gg of total RNA was reverse transcribed using the Reverse Transcription System (Promega Corp., Madison, Wis.). A fraction of the resulting cDNA was used as a template for PCR amplification. Forty cycles of PCR were conducted (95° C., 30 sec, 50 ° C., 30 sec, and 72 ° C., 60 sec) using specific primers. The specific primer sequences used were as follows: 1) c-raf primers: 5′-TCAGAGAAGCTCTGCTAAG-3′ and 5′-CAATGCACTGGACACCTTA-3′; 2) actin primers: 5′-ACCTTCTACAATGAGCTGCG-3′ and 5′-GCTTGCTGATCCACATCTGC-3′. Actin was used as housekeeping gene control. Total RNA, isolated from cells transfected with either control pssXD-I or pssXD-II containing c-raf DNA enzyme sequence, was reverse transcripted and PCR amplified using a pair of c-raf specific primers. PCR amplification of actin rnRNA was used as a control to normalize loading quantity among different samples. As shown in FIG. 15, a significant reduction (approximately 70-80%) of c-raf mRNA was detected in the cells transfected with pssXD-II (Lane 2) compared to teat of control (Lane 1).

The levels of c-raf protein-in A549 cells transfected with pssXD-I or pssXD-II were assessed by Western Blot analysis. 30 μg of cell extracts were subjected to electrophoresis on a 12% sodium dodecyl sulfate-polyacrylamide gel (SDS-PAGE). Proteins were electrotransferred using a Mini Trans-Blot Electrophoretic Transfer Cell according to the manufacture's instructions (BioRad Laboratories, Hercules, Calif.) to a Hybond ECL membrane (Amersham Pharmacia Biotec, Piscataway, N.J.). The membrane was subsequently blocked in a buffer containing 25 mM Tris-HCl, pH 7.5, 500 mM NaCl, 0.05% Tween-20, and 5% non-fat milk and then incubated with primary and HRP-conjugated secondary antibodies for 45 min each. The polyclonal antibodies (anti-raf1) against c-raf and monoclonal antibodies (Ab-1) against actin were purchased from Calbiochem-NovaBiochem Corp. (San Diego, Calif.). Monoclonal antibodies (IgG1, C-2-10) against poly-ADP ribose polymerase (PARP) were purchased either from Clontech Laboratories, Inc. (Palo Alto, Calif.). Proteins were visualized using SuperSignal West Pico Chemiluminescent Substrate Kit (Pierce, Rockford, Ill.). As shown in FIG. 16, the level of c-raf protein in control pssXD-I transfected cells (Lane 2) was similar to that of untransfected cells (Lane 3). However, cells transfected with pssXD-II expressing c-raf DNA enzyme (Lane 1) had lower protein levels (approximately 20-30%) of c-raf compared to the controls.

Two standard apoptosis assays, genomic DNA cleavage and PARP cleavage, were-performed to determine whether expression of c-ra! DNA enzyme could induce A549 cell apoptosis. Genomic DNA cleavage was determined using a LM-PCR Ladder Assay Kit (Clontech Laboratories, Inc., Palo Alto, Calif.) according to the manufacturer's instructions. Briefly, 0.5 μg of genomic DNA was ligated to adaptors, supplied by Clontech Laboratories, Inc. with T4 DNA ligase overnight at 15° C. A fraction of adaptor-ligated. DNA was used as template in LM-PCR. Twenty-five cycles of PCR (95° C., 1 min and 72 ° C., 3 min) with an extension of 15 min at 72° C. were conducted. Genomic DNA, isolated from cells transiently transfected with either pssXD-I (control) or pssXD-II (DNA enzyme), was ligated to specific adaptors. Subsequently, LM-PCR was carried out using a c-raf primer and a specific primer. As shown in FIG. 17, there was a significant increase in fragmented genomic DNA in cells transfected with pssXD-II (Lane 1) compared to cells transfected with pssXD-I (control) (Lane 2), or untransfected cells (Lane 3). These results suggest that the increase in fragmented genomic DNA is a result of DNA cleavage caused by suppression of c-raf gene expression altered by the presence of the c-raf DNA enzyme.

Another apoptosis assay, the PARP cleavage assay, was conducted using Western Blot analysis. Compared to the controls (Lanes 2-3), cells transfected with pssXD-II (Lane 1) had decreased amounts of full-length PARP (FIG. 18), again indicating induction of cell apoptosis by suppression of c-raj gene. Similar amounts of protein were loaded per lane as determined by the presence of actin (Lanes 1-3).

Use of the β-gal reporter gene as a target facilitates convenient measurement of the alteration of gene function by β-galactosidase activity assay. As shown in FIG. 19, twenty-four hours after co-transfection into A549 lung carcinoma cells, the DNA enzyme targeting the β-gal protein translation starting site (ATG) included in the pssXE plasmid reduced β-galactosidase activity by approximately 75%. A mutated DNA enzyme sequence produced intracellularly had little effect on β-galactosidase activity (also shown in FIG. 17).

The experiments described above demonstrate the use of an expression vector for production of ssDNA in vitro and in vivo by multiple stepwise reactions using eukaryotic RT reactions and various cDNA priming reactions that successfully altered gene function in vivo. Those skilled in the art will recognize that the present invention is not limited to these specific embodiments. It will be recognized, for instance, that many nucleic acid sequences may be utilized depending upon the specific target and/or is mode of inhibitory action of the SOI. Similarly, the SOI may be located in either or both of the two positions, e.g., between the IR and/or between the PBS and the 3′ aspect of the IR. Likewise, the SOI may or may not include a DNA enzyme sequence depending upon the particular target and/or mode of action of the SOI and/or the DNA enzyme sequence. Those skilled in the art who have the benefit of this disclosure will recognize that any desired therapeutic effect is produced by this method by transfecting the appropriate SOI into a eukaryotic cell using the vector system of the present invention. By way of example, and not limitation, the following excitatory and inhibitory nucleic acid sequences are known in the art and may be utilized as the SOI to alter gene expression when incorporated into an expression vector constructed in accordance with the teachings of the present invention:
Sequences that act as antisense oligonucleotides to one or more RNA molecules encoding one of the several dopamine receptors for therapy of Parkinson's disease. The antisense oligonucleotides bind specifically to expression-controlling sequences of such RNA molecules, thereby selectively controlling expression of one or more doparine receptor subtypes, and alleviating the pathological conditions related to their expression;
Sequences that inhibit expression of KSHV virion protein 26, including sequences that act as antisense and/or triplex oligonucleotides for treatment of Karposi's syndrome as described in U.S. Pat. No. 5,856,903;
Oligonucleotides for control of the expression of IL-8 and/or IL-8 receptor to control growth, metastasis and/or angiogenesis in tumors as described in U.S. Pat. No. 5,856,903;
Oligonucleotides having a sequence of nucleotide bases specifically hybridizable with a selected sequence of a cytomegalovirus DNA or RNA, specifically, sequences targeting cytomegalovirus DNA or RNA coding for the IE1, IE2, or DNA polymerase proteins. It is preferred that such oligonucleotides have between about 5 and about 50 nucleic acid base units as described in U.S. Pat. No. 5,442,049,
Oligonucleotides specifically hybridizable with RNA or DNA deriving from a gene corresponding to one of the open reading frames UL5, UL8, UL9, UL20, UL27, UL29, UL30, UL42, UL52 and IE175 of herpes simplex virus type 1 comprising nucleotide units sufficient in identity and number to effect such specific hybridization. It is preferred that the oligonucleotides be specifically hybridizable with a translation initiation site, coding region or 5′ untranslated region. The oligonucleotides are designed to be specifically hybridizable with DNA, or preferably, RNA from one of the species herpes simplex virus type 1 (HSV-1), herpes simplex virus type (HSV-2), cytomegalovirus, human herpes virus 6, Epstein Barr virus (EBV) or varicella zoster virus (VZV). Such oligonucleotides are conveniently and desirably presented as a pharmaceiitical composition in a pharmaceutically acceptable carrier as described in U.S. Pat. No. 5,514,577. Persons skilled in the art will recognize that the particular open reading frames described for herpes simplex virus type 1 find counterparts in the other viruses named. Thus each of herpes simplex virus type 2, cytomegalovirus, human herpes virus type 6, Epstein Barr virus and varicella zoster virus are believed to have many analogous open reading frames which code for proteins having similar functions. Accordingly, the present invention is directed to antisense oligonucleotide therapy in which the oligonucleotides are directed to any of the foregoing viruses, or indeed to any similar viruses which may become known hereafter, which have one or more of such analogous open reading frames. For convenience in connection with the present invention, all such viruses are denominated as herpes viruses;
Antisense oligonucleotides to proto-oncogenes, and in particular to the c-myb gene, for use as antineoplastic and immunosuppressive agents as described in U.S. Pat. No. 5,098,890;
Antisense oligonucleotides against ICAM-1 gene expression in interleukin-1 beta-stimulated cells for use as anti-inflammatory agents with activity towards a variety of inflammatory diseases or diseases with an inflammatory component such as asthma, rheumatoid arthritis, allograft rejections, inflammatory bowel disease, various dermatological conditions, and psoriasis. In addition, inhibitors of ICAM-1, VCAM- 1, and ELAM-1 may be effective in the treatment of colds due to rhinovirus infection, AIDS, Kaposi's sarcoma and some cancers and their metastasis as described in U.S. Pat. No. 5,843,738. Similarly, International Application No. PCT/US90/02357 discloses DNA sequences encoding endothelial adhesion molecules (ELAMs), including ELAM-1 and VCAM-1 and VCAM-1b. The oligonucleotides designated ISIS 1570 and ISIS 2302 are specifically contemplated as being used as the sequence of interest in the method of the present invention for decreasing the metastatic potential of target cells; and
Protein-binding oligonucleotides (aptamers) that specifically bind target molecules such as proteins, and particularly thrombin, in the host cell as described in U.S. Pat. No. 5,840,867. These non-oligonucleotide target molecules bind nucleic acids (Blackwell, T.K., el al., 250 Science 1104-1110 (1990); Blackwell, T. K., et al., 250 Science 1149-1152 (1990); Turek, C. and

L. Gold, 249 Science 505-510 (1990); Joyce, G. F., 82 Gene 83-87 (1989)), specifically controlling the biological activity of the protein.

This list of inhibitory and excitatory sequences is not intended to be all-inclusive. It does not, for instance, list the AG30 TFO described above that has not only been produced in vivo, but shown to function in vivo to bind in triplex fashion and induce genomic recombination in mouse FL-10 cells. Surprisingly, not only did this particular ssDNA induce recombination in the host cells, but recombinants were produced at a frequency 7-fold higher than stimulated by a synthetic AG30 TFO that was transfected into the host cells under the same conditions as the expression vector of the present invention. In short, it is envisioned that expression vectors can be constructed in accordance with the present invention that are capable of tranfection into any host cell for producing a sequence that can target any, or at least a wide variety, of genes and/or the control functions for the genes to alter the function of the target gene(s).

Although described with reference to the figures and specific examples set out herein, those skilled in-the art will recognize that certain changes can be made to the specific elements set out herein without changing the manner in which those elements function to achieve their intended respective results. For instance, the cassette described herein is described as comprising three genetic elements, a sequence of interest, a primer binding sequence, and a tandem inverted repeat, and when transfected into a target cell with a reverse transcriptase gene under control of a suitable promoter, produces the inhibitory nucleic acid sequence described herein. However, those skilled in the art will recognize that, for instance, the mouse Moloney leukemia virus reverse transcriptase gene described for use as the reverse transcriptase gene of the cassette can be replaced with other reverse transcriptase genes (the reverse transcriptase gene from human immunodeficiency virus was one such gene which was noted above) and that promoters other than the CMV promoter described herein may be used to advantage. As noted above, the stem-loop intermediate that is formed may or may not include a restriction endonuclease site and its susceptibility to denaturation is manipulated to advantage depending upon the particular sequence of interest that is intended to be produced from tha, intermediate. All such changes, and others that will be made clear to those skilled in the art by this description modifications which do not depart from the spirit of the present invention, are intended to fall within the scope of the following claims.

| | | |
|---|---|---|
| 1. | Name: 3′-RT/Mol-Hind III | (24-mer) |

Sequence: 5′-CTT GTG CAC AAG CTT TGC AGG TCT-3′

| | | |
|---|---|---|
| 2. | Name: 5′-RT/Mol-Sac I | (32-mer) |

Sequence: 5′-GGG ATC AGG AGC TCA GAT CAT GGG ACC
AAT GG-3′

| | | |
|---|---|---|
| 3. | Name: 5′-Mbo II-Hind III | (30-mer) |

Sequence: 5′-CAA TTA AGG AAA GCT TTG AAA AAT TAT
GTC-3′

| | | |
|---|---|---|
| 4. | Name: 5′-RT-Not-Mbo-Link | (129-mer) |

Sequence: 5′-CTA GGT CGG CGG CCG CGA AGA TTG GTG
CGC ACA CAC ACA ACG CGC ACC AAT CTT CGC GGC CGC
CGA CCC GTC AGC GGG GGT CTT TCA TTT GGG GGC TCG
TCC GGG ATC GGG AGA CCC CTG CCC AGG GCC

| | | |
|---|---|---|
| 5. | Name: 3′-RT-Not-Mbo-Link | (121-mer) |

Sequence: 5′-CT GGG CAG GGG TCT CCC GAT CCC GGA
CGA GCC CCC AAA TGA AAG ACC CCC GCT GAC GGG TCG
GCG GCC GCG AAG ATT GGT GCG CGT TGT GTG TGT GCG
CAC CAA TCT TCG CGG CCG CCG AC-3′

| | | |
|---|---|---|
| 6. | Name: 5′-Nhe-Sac-Link | (18-mer) |

Sequence: 5′-CTA GCG GCA AGC GTA GCT-3′

| | | |
|---|---|---|
| 7. | Name: 3′-Nhe-Sac-Link | (10-mer) |

Sequence: 5′-ACG CTT GCC G-3′

| | | |
|---|---|---|
| 8. | Name: 3′-Mbo II-Xba I | (27-mer) |

Sequence: 5′-TAA TGG CCC GGG CAT AGT CGG GTA
GGG-3′

| | | |
|---|---|---|
| 9. | Name: 5′-Hind-link-Histag | (43-mer) |

Sequence: 5′-A GCT GGA TCC CCC GCT CCC CAC CAC CAC
CAC CAC CCT GCC CCT-3′

| | | |
|---|---|---|
| 10. | Name: 3′-Hind-link-Histag | (42-mer) |

Sequence: 5′-AGC AGG GGC AGG GTG GTG GTG GTG GTG
GGG AGC GGG GGA TCC-3′

| | | |
|---|---|---|
| 11. | Name: 5′-Not-link-test1 | (57-mer) |

Sequence: 5′-G GCC GGA AGA TTG GGG CGC CAA AGA GTA
ACT CTC AAA GGC ACG CGC CCC AAT CTT CC-3′

| | | |
|---|---|---|
| 12. | Name: 3′-Not-link-test1 | (57-mer) |

Sequence: 5′-GGC CGG AAG ATT GGG GCG CGT GCC TTT
GAG AGT TAC TCT TTG GCG CCC CAA TCT TCC-3′

| | | |
|---|---|---|
| 13. | Name: 5′-Not-Mbo-link-telo | (92-mer) |

Sequence: 5′-GGC CGG AAG ATT GGG GCG TTA GGG TTA
GGG TTA GGG TTA GGG TTA GGG TTA GGG TTA GGG TTA
GGG TTA GGG TTA GGG CGC CCC AAT CTT CC-3′

| | | |
|---|---|---|
| 14. | Name: 3′-Not-Mbo-link-telo | (92-mer) |

Sequence: 5′-GGC CGG AAG ATT GGG GCG CCC TAA CCC
TAA CCC TAA CCC TAA CCC TAA CCC TAA CCC TAA CCC
TAA CCC TAA CCC TAA CGC CCC AAT CTT CC-3′

| | | |
|---|---|---|
| 15. | 5′-SL-linker-Fok1-RT | (111-mer) |

Sequence: 5′-CTA GTC GGA TGC GGC CGC TGC ACA ACA
ACA CAC AAC ACA GCG GCC GCA TCC GAT CAG CGG GGG
TCT TTC ATT TGG GGG CTC GTC CGG ATC GGG AGA CCC
CTG CCC AGC GCC-3′

| | | |
|---|---|---|
| 16. | 3′-SL-linker-Fok1-RT | (103-mer) |

Sequence: 5′-CTG GGC AGG GGT CTC CCG ATC CGG ACG
AGC CCC CAA ATG AAA GAC CCC CGC TGA TCG GAT GCG
GCC GCT GTG TT_{G} TTT GTT GTT GTG CAG CGG CCG CAT
CCG A-3′

| | |
|---|---|
| 17. | Name: XmaI-BglII-Stop 1 |

Sequence: 5′-CCGGATCTAGACCGCAAGCTTCATTTAAA-3′

| | |
|---|---|
| 18. | Name: XmaI-BrlII-Stop 2 |

Sequence: 5′-GATCTTTAAATGAAGCTTGCGGTCTCGAT-3′

| | |
|---|---|
| 19. | Name: 5′-E/S/P/P-LINKER |

Sequence: 5′TCGAGCGGCCAGGGGTCTCCCGATCCCGGACGAGCCCC
CAAAGAATTCCGCGGCTGCAGTTAAT-3′

| | |
|---|---|
| 20. | Name: 3′-E/S/P/P-LINKER |

Sequence: 5′-TAACTGCAGCCGCGGAATTCTTTGGGGGCTCGTCCGG
GATCGGGAGACCCCTGGCCGC-3′

1. An expression vector for producing a single stranded sequence of nucleic acids for altering expression of an endogenous nucleic acid target sequence when delivered to a host cell comprising:
a cassette comprised of a sequence of interest flanked by inverted tandem repeats and a 3′ primer binding site (PBS), the sequence of interest being comprised of a nucleic acid sequence designed to produce a single stranded sequence of nucleic acids that binds to an endogenous nucleic acid sequence when reverse transcribed; and
a reverse transcriptase/RNase gene.

2. The expression vector of claim 1 additionally comprising a restriction endonuclease gene.

3. The expression vector of claim 1 additionally comprising a restriction endonuclease. site formed by pairing of the sequence comprising the inverted tandem repeat.

4. The expression vector of claim 1 wherein said reverse transcriptase gene is under control of an inducible promoter.

5. The expression vector of claim 1 wherein said reverse transcriptase gene is promoted with a eukaryotic promoter.

6. The expression vector of claim 1 additionally comprising a eukaryotic promoter for said sequence of interest.

7. The expression vector of claim 6 wherein the promoter for said sequence of interest is selected from the group of promoters comprising constitutive, inducible, wide-spectrum, or tissue specific promoters.

8. The expression vector of claim 1 additionally comprising a second sequence coding for a sequence of interest between the PBS and the inverted tandem repeat.

9. A plasmid including the expression vector of claim 1.

10. A host cell transfected with the expression vector of claim 1.

11. The plasmid pssXE.
